(19) United States
(12) Patent Application Publication
(10) Pub. No.: US 2023/0186495 A1
(43) Pub. Date: Jun. 15, 2023

(54) PRE-MORBID CHARACTERIZATION OF ANATOMICAL OBJECT USING ORTHOPEDIC ANATOMY SEGMENTATION USING HYBRID STATISTICAL SHAPE MODELING (SSM)

(71) Applicant: Howmedica Osteonics Corp., Mahwah, NJ (US)

(72) Inventors: Sergii Poltaretskyi, Ependes FR (CH); Charlotte Le Saint, Brest (FR); Jean Chaoui, Locmaria Plouzané (FR)

(73) Assignee: Howmedica Osteonics Corp., Mahwah, NJ (US)

(21) Appl. No.: 17/923,191

(22) PCT Filed: Apr. 7, 2021
(86) PCT No.: PCT/US2021/026191

§ 371 Date: Nov. 3, 2022

Related U.S. Application Data
(60) Provisional application No. 63/021,337, filed on May 7, 2020.

(51) Int. Cl. G06T 7/35; G06T 19/20; G06T 7/55; A61B 34/10

(52) U.S. Cl. CPC G06T 7/35; G06T 19/20; G06T 7/55; A61B 34/10; G06T 2207/20124; G06T 2207/30052; G06T 2207/30008; G06T 2219/2021; G06T 2219/2004; G06T 2210/44; G06T 2210/41; A61B 2034/105; A61B 2090/365

(57) ABSTRACT

Techniques are described for determining a pre-morbid shape of an anatomical object. A method includes receiving first image data of a first anatomical structure and second image data of a second anatomical structure. The first and second anatomical structures are anatomically related. The method includes determining a first shape model based on the first image data and a joint statistical shape model (SSM). The method includes determining a second shape model based on the first shape model, the first image data, and the second image data, the second shape model including a second estimated shape of the first anatomical structure and a second estimated shape for the second anatomical structure. The method includes generating anatomical information indicative of the pre-morbid shape of at least the second anatomical structure based on the second shape model.

This application claims the benefit of U.S. Provisional Patent Application No. 63/021,337, filed 7 May 2020, the entire contents of which is incorporated herein by reference.

### BACKGROUND

Surgical joint repair procedures involve repair and/or replacement of a damaged or diseased joint. A surgical joint repair procedure, such as joint arthroplasty as an example, may involve replacing the damaged joint with a prosthetic that is implanted into the patient's bone. Proper selection or design of a prosthetic that is appropriately sized and shaped and proper positioning of that prosthetic are important to ensure an optimal surgical outcome. A surgeon may analyze damaged bone to assist with prosthetic selection, design and/or positioning, as well as surgical steps to prepare bone or tissue to receive or interact with a prosthetic.

### SUMMARY

This disclosure describes example techniques to perform pre-morbid characterization of patient anatomy, such as an anatomical object where information (e.g., derived from image data) to characterize the anatomical object is missing. Pre-morbid characterization refers to determining anatomical information that predicts characteristics (e.g., size, shape, location) of patient anatomy as the anatomy existed prior to damage to the patient anatomy or disease progression of the anatomy. In examples described in this disclosure, the anatomical information may be a graphical shape model, such as a 3D volume, that a surgeon can view to assist in planning of an orthopaedical surgical procedure, e.g., to repair or replace an orthopedic joint, as one example.

In some examples, generating the pre-morbid characterization relies on imaging of the anatomical objects after the damage or disease progression. However, the damage or disease progression causes loss of portions of the anatomical objects that would be desirable to have for generating the pre-morbid characterization. Additionally, the nature of the lost portions of the anatomical object may result in a situation where there is not enough information to determine a anatomical information. For example, damage to a humerus of a patient may prevent a anatomical information from being selected because there may not be enough information to determine a length of a shaft of the humerus. In some examples, because of the nature of soft tissue around the anatomical object, available images of the anatomical object may be such that it is difficult to distinguish boundaries of the anatomical object using images processing techniques (e.g., without performing more invasive imaging, etc.). For example, certain contours of a scapula may be difficult to distinguish in a standard CT or X-ray image. As another example, imaging modalities may be imperfect and cause loss of image fidelity such that determining the contours of an anatomical object, such as a scapula, may be difficult.

This disclosure describes example techniques to determine a representation of a pre-morbid anatomical object (e.g., a predictor of the pre-morbid anatomical object) using hybrid statistical shape modeling (SSM), and particularly, determining a shape model representative of the patient pre-morbid anatomical object based on a current state of the anatomical object of interest and a related anatomical object, in accordance with a cost function. A hybrid SSM model is a shape model that includes representations of multiple different related anatomical objects. In some examples, a hybrid SSM may be referred to as a “joint SSM” (e.g., when anatomical objects a related based on a joint connection). Anatomical objects are related when at least some characteristics of a first anatomical object can be predicted and/or estimated based on information from a second anatomical object.

A example method includes receiving first image data of a first anatomical structure and second image data of a second anatomical structure. The first and second anatomical structures are anatomically related. The method includes determining a first shape model based on the first image data and a joint statistical shape model (SSM). The first shape model includes a first estimated shape of the first anatomical structure and a first estimated shape for the second anatomical structure. The joint SSM is a shape model of combined first and second anatomical structures. The method includes determining a second shape model based on the first shape model, the first image data, and the second image data, the second shape model including a second estimated shape of the first anatomical structure and a second estimated shape for the second anatomical structure. The method includes generating anatomical information indicative of the pre-morbid shape of at least the second anatomical structure based on the second shape model.

An example apparatus includes memory storing a plurality of first shape models, and processing circuitry. The processing circuitry receives first image data of a first anatomical structure and second image data of a second anatomical structure. The first and second anatomical structures are anatomically related. The processing circuitry determines a first shape model based on the first image data and a joint statistical shape model (SSM). The first shape model includes a first estimated shape of the first anatomical structure and a first estimated shape for the second anatomical structure. The joint SSM is a shape model of combined first and second anatomical structures. The processing circuitry determines a second shape model based on the first shape model, the first image data, and the second image data, the second shape model including a second estimated shape of the first anatomical structure and a second estimated shape for the second anatomical structure. Additionally, the processing circuitry generates anatomical information indicative of the pre-morbid shape of at least the second anatomical structure based on the second shape model.

The details of various examples of the disclosure are set forth in the accompanying drawings and the description below. Various features, objects, and advantages will be apparent from the description, drawings, and claims.

### BRIEF DESCRIPTION OF DRAWINGS

FIG. 1 is a block diagram illustrating an example computing device that may be used to implement the techniques of this disclosure.

FIG. 2A illustrates an example mean shape model that is modified in accordance with one or more example techniques described in this disclosure.

FIG. 2B illustrates an example intermediate shape model formed by modifying the mean shape model of FIG. 2A with image data of a first anatomical object in accordance with one or more example techniques described in this disclosure.

FIG. 2C illustrated an example final shape model formed by modifying the intermediate shape model of FIG. 2B with image data of a second anatomical object anatomically related to the first anatomical object in accordance with one or more example techniques described in this disclosure.

FIG. 3 is a flowchart illustrating an example method of operation in accordance with one or more example techniques described in this disclosure.

FIG. 4 is an illustration of a scapula having points of interest used to determine a coordinate system of a patient.

FIGS. 5A and 5B are illustrations of a planar cut through a scapula for determining a coordinate system of a patient.

FIG. 6 is a conceptual diagram of a perspective view of a normal line to a plane through a scapula for determining a coordinate system of a patient.

FIG. 7 is a conceptual diagram of another perspective view of a normal line to a plane through a scapula for determining a coordinate system of a patient.

FIG. 8 is a conceptual diagram illustrating a transverse axis through a scapula for determining a coordinate system of a patient.

FIGS. 9A and 9B are conceptual diagrams illustrating an example of sagittal cuts through a scapula for determining the transverse axis of FIG. 8.

FIGS. 10A-10C are conceptual diagrams illustrating results of sagittal cuts through scapula for determining the transverse axis of FIG. 8.

FIGS. 11A and 11B are conceptual diagrams illustrating another example of sagittal cuts through a scapula for determining the transverse axis of FIG. 8.

FIG. 12 is a conceptual diagram illustrating a transverse axis through a scapula and a normal line to a plane through the scapula for determining a coordinate system of a patient.

FIG. 13 is a conceptual diagram illustrating movement of a transverse axis through a scapula and a normal line to a plane through the scapula to a central location of a glenoid for determining a coordinate system of a patient.

FIG. 14 is a conceptual diagram illustrating an example of an initial alignment of a segmentation object to a shape model.

FIG. 15 is a conceptual diagram illustrating an example of an intermediate alignment of a segmentation object to a shape model.

FIG. 16 is a conceptual diagram illustrating an example for determining difference values for iterative closest point (ICP) algorithm.

FIGS. 17A and 17B are conceptual diagrams illustrating portions of a glenoid for determining parameters of a cost function used to determine a pre-morbid shape of anatomy of a patient.

FIG. 18 is a flowchart illustrating an example method of operation in accordance with one or more example techniques described in this disclosure.

FIG. 19 is a flowchart illustrating an example method of operation in accordance with one or more example techniques described in this disclosure.

FIG. 20 is a flowchart illustrating an example method of operation in accordance with one or more example techniques described in this disclosure.

FIGS. 21A-21H represent examples of initial alignment of a diaphysis with a humerus statistical shape model (SSM).

FIGS. 22A-22C show examples of humerus shape estimation based on dispahysis shape.

### DETAILED DESCRIPTION

A patient may suffer from a disease (e.g., aliment) that causes damage to the patient anatomy, or the patient may suffer an injury that causes damage to the patient anatomy. For shoulders, as an example of patient anatomy, a patient may suffer from primary glenoid humeral osteoarthritis (PGHOA), rotator cuff tear arthropathy (RCTA), instability, massive rotator cuff tear (MRCT), rheumatoid arthritis (RA), post-traumatic arthritis (PTA), osteoarthritis (OA), or acute fracture, as a few examples.

To address the disease or injury, a surgeon may perform a surgical procedure such as Reversed Arthroplasty (RA), Augmented Reverse Arthroplasty (RA), Standard Total Shoulder Arthroplasty (TA), Augmented Total Shoulder Arthroplasty (TA), or Hemispherical shoulder surgery, as a few examples. There may be benefits for the surgeon to determine, prior to the surgery, characteristics (e.g., size, shape, and/or location) of the patient anatomy. For instance, determining the characteristics of the patient anatomy may aid in prosthetic selection, design and/or positioning, as well as planning of surgical steps to prepare a surface of the damaged bone to receive or interact with a prosthetic. With advance planning, the surgeon can determine, prior to surgery, rather than during surgery, steps to prepare bone or tissue, tools that will be needed, sizes and shapes of the tools, the sizes and shapes or other characteristics of one or more protheses that will be implanted and the like.

As described above, pre-morbid characterization refers to characterizing the patient anatomy as it existed prior to the patient suffering disease or injury. However, pre-morbid characterization of the anatomy is generally not available because the patient may not consult with a doctor or surgeon until after suffering the disease or injury. In some cases, it may be possible to use one side of the patient (e.g., shoulder on one side) as representative of the pre-morbid characterization of the other side (e.g., shoulder on other side). However, the disease could be bilateral, meaning that there is impact on both sides. Also, the patient anatomy may not be symmetric (e.g., shoulders are not symmetric) and may not reflect the pre-morbid characteristics of the contralateral side.

In some techniques, the image data of a first anatomical object (e.g., a pathological anatomical object) is compared to shape models generated from a statistical shape model of that anatomical object to generate a pre-morbid shape. However, in such techniques, while the resulting pre-morbid shape might generally be accurate, a higher degree of accuracy may be available. For example, because of lack of image data for the first anatomical object, including parts that are missing due to injury or disease, the resulting pre-morbid shape is imperfect. In such examples, the first anatomical object may be damaged such that a highly accurate model of the diseased first anatomical object may not be possible relying solely on information derived from images of that first anatomical object. For example, the diaphysis of the humerus may be damaged such that everything above the surgical neck is missing. As such an example, it may not be possible to select an estimated model of the humerus with the correct length of the diaphysis. As another example, obtaining images of some anatomical objects to convert into data suitable for modeling may be difficult using image processing technique because of lack of clear definitions in images between, for example, bone and soft tissue. As such an example, it may be difficult to obtain data from images of a scapula because of the surrounding soft tissue may obscure the contours of some portions of the scapula. Using additional image data of related anatomical object (e.g., a “second anatomical object”), where more image data is available, a computer device may derive an intermediate shape model that includes both the first anatomical object and the second anatomical object from a hybrid SSM. In some examples, one of the anatomical object may be pathological and the other anatomical object may be either pathological or non-pathological. Subsequently, a final model may be generated based on the intermediate shape model at least using the available image data from the first anatomical object.

Techniques are described below to determine a representation of a pre-morbid anatomical object using a hybrid statistical shape model (SSM) based on statistical shape model(s) of related anatomical object(s). A hybrid statistical shape model (SSM) may be a model generated from determining a mean shape from participants in a study. Using patient demographic information and various measurements extracted from the segmented image data, a computing device may select or generate the appropriate shape model (e.g., an initial shape model) for the patient. In some examples, the SSM may be constant for all patients, rather than generating an appropriate shape model for a particular patient. The shape model may be represented as a point cloud or a function with parameters for generating a point cloud.

A computer device may determine an intermediate shape model for a first anatomical object and a second anatomical object using image data of the first anatomical object and an initial shape model (e.g., hybrid SSM). The two anatomical objects are orthopedically related such that the shape of one anatomical object is predictive of at least a portion of the shape of the other anatomical object. For example, the first anatomical object may be a scapula and the second anatomical object may be a humerus, or vice-versa. In some examples, the first anatomical object is the object for which there is more complete image data. In the above example where a diseased humerus is missing parts of the humerus, the intermediate shape model including both the scapula and the humerus may be generated based on image data from the scapula. A computer device determines a final shape model using the available data of the second anatomical object. A computer device predicts the pre-morbid shape model by fitting the available data of the second anatomical object to the intermediate shape model.

Pre-morbid anatomy, also called native anatomy, refers to the anatomy prior to the onset of a disease or the occurrence of an injury. Even after disease or injury, there may be portions of the anatomy that are healthy and portions of the anatomy that are not healthy (e.g., diseased or damaged). The diseased or damaged portions of the anatomy are referred to as pathological anatomy, and the healthy portions of the anatomy are referred to as non-pathological anatomy.

In some examples, techniques described below are used to determine a representation of related anatomical objects (e.g., predictors of the pre-morbid anatomical objects) using hybrid statistical shape models (SSM), and particularly, determining a shape model representative of the both anatomical objects in accordance with a cost function. For example, the disclosure describes aligning a segmented shape representing both anatomical objects to a coordinate system of an initial shape model (e.g., hybrid SSM). The initial shape model includes two related anatomical objects. For instance, a coordinate system of segmented shape is aligned to the coordinate system of the initial shape model so that the segmented shape is defined in the same space as the initial shape model. The result of the aligning is an aligned shape. In some examples, when aligning the coordinate system of segmented shape to the coordinate system of the initial shape mode, the segmented shape based on image data of both anatomical objects is used.

After aligning the segmented shape of both anatomical objects to the coordinate system of the initial shape model (e.g., hybrid SSM), the computing device may deform the initial shape model through an iterative process to register the initial shape model. This registration may include adjusting the size and shape of the representations of both anatomical objects in the initial shape model to fit a first anatomical object (e.g., the non-pathological anatomical object and/or anatomical object for which there is sufficient, reliable image data). That is, during this registration, adjusting the representation of the first anatomical object in the initial shape model to fit the first anatomical object may proportionally adjust the size and shape of the second anatomical object in the initial shape model (e.g., where the second anatomical object may be the pathological anatomical object and/or anatomical object for which there is not sufficient, reliable image data). The result of the registration may be an intermediate shape model that includes a first approximation of the size and shape of the first anatomical object and a first approximation of the pre-morbid size and shape of the second anatomical object.

Subsequently, the intermediate shape model is modified in an iterative process to register the intermediate shape model. This registration may include adjusting the size and shape of the representations of both anatomical objects in the intermediate shape model to fit the available data of at least the second anatomical object (e.g., pathological anatomical object and/or anatomical object for which there is not sufficient, reliable image data). During this registration, data of both anatomical objects may be used to modify the shape and size of the representations of the anatomical objects in the intermediate shape model so that the representation of the first anatomical object fits the data of the first anatomical object and the representation of the second anatomical object fits the available data of the second anatomical object. The result of the registration of the intermediate shape model may be the final shape model. The final shape model is a final approximation of the non-pathological anatomical object and an approximation of the pre-morbid shape of the pathological anatomical object.

For example, assume that the patient anatomical objects include an injured or diseased scapula (e.g., pathological scapula) and a non-injured humerus (e.g., non-pathological humerus). In this example, the initial shape model is a hybrid SSM that includes both anatomical objects (e.g., a non-pathological scapula and a non-pathological humerus). With the example techniques described in this disclosure, the intermediate shape model is generated from the hybrid SSM using the image date of the non-pathological humerus. The intermediate shape model is modified to generate the final shape model using image data of the pathological scapula (and, in some examples, the non-pathological humerus). In this example, the non-pathological scapula of the final shape model is an approximation of the pre-morbid size and shape of the patient scapula. Likewise, the non-pathological humerus of the final shape model is an approximation of the size and shape of the patient humerus.

The above example describes an initial shape model, an intermediate shape model, and a final shape model. In some examples there may be a plurality of intermediate shape models based on implementation or based on amount of anatomical objects that are used for the initial shape model (e.g., if three anatomical objects form the initial shape model, then there may be two or more intermediate shape models). Accordingly, even though the example techniques described in this disclosure describe an intermediate shape model being generated from an initial shape model, and a final shape model generated from the intermediate shape model, the example techniques should not be considered limited to cases where there is only one intermediate shape model. Rather, in this disclosure, intermediate shape model may be considered as an example of shape model that is generated from the initial shape model and that is used to generate the final shape model, with the possibility of there being other shape models between the initial and intermediate shape model and between the intermediate and final shape model.

As described above, a computing device may align the segmented shape (e.g., a representation of both of the anatomical objects) to the initial shape model. To perform the alignment, the computing device may determine a coordinate system for the segmented shape. Because at least one of the anatomical object includes pathological anatomy that is diseased or damaged, it is possible that portions of the pathological anatomy, used for determining a coordinate system, are no longer present in the patient. Also, registration (e.g., deformation of the shape model to register the shape model to aligned shape) may be limited to comparison of the shape model to the non-pathological anatomy. Moreover, imaging techniques may not properly capture the non-pathological anatomy. For instance, based on scans performed on the patient, such as computed tomography (CT) scans or radiographs such as x-ray, MRI, and ultrasound, a computing device may generate image data of the patient anatomy. In under-segmentation, it is possible that certain portions of the patient anatomy (e.g., scapula) are not fully captured, and there are missing portions (e.g., in this case, a resulting segmented object does not include the entire actual anatomical object). This under-segmentation could be due to types of machines used to scan the patient, noise in the scans, bone-to-bone contact, etc. In some cases, the radiologist tries to minimize patient's exposure to irradiation and stops the scanner once the anatomical object of interest is reached. For instance, the radiologist may stop scanning the patient anatomy when the glenoid is reached. In such cases, image information for other anatomical objects may be lost (e.g., lose the inferior and medial parts of the scapula). In such cases, it is beneficial to use a hybrid SSM as describe below to obtain a representation of the pre-morbid state of an anatomical object.

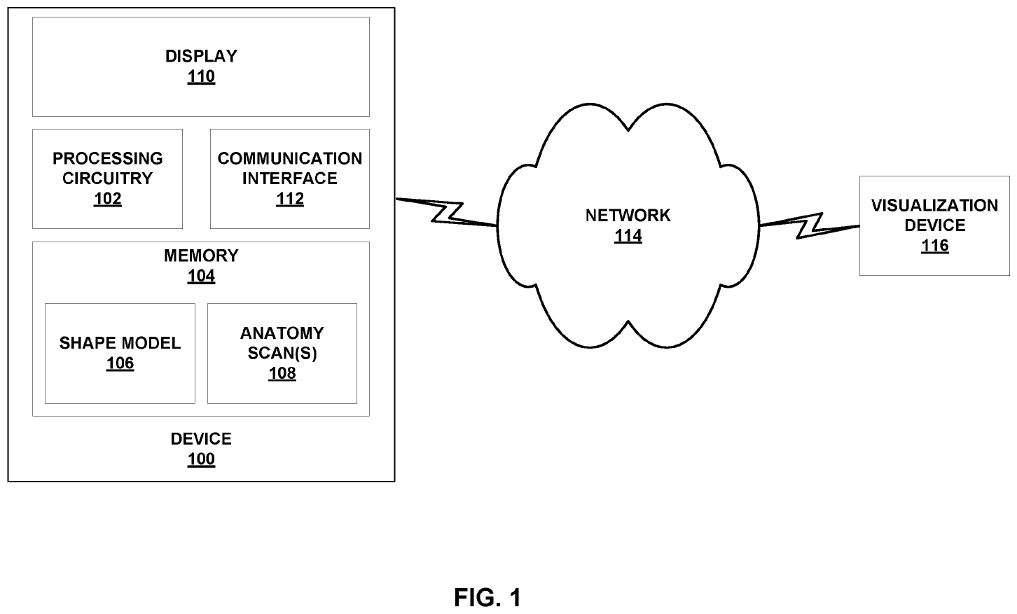

FIG. 1 is a block diagram illustrating an example computing device that may be used to implement the techniques of this disclosure. FIG. 1 illustrates device 100, which is an example of a computing device configured to perform one or more example techniques described in this disclosure.

Device 100 may include various types of computing devices, such as server computers, personal computers, smartphones, laptop computers, and other types of computing devices. Device 100 includes processing circuitry 102, memory 104, and display 110. Display 110 is optional, such as in examples where device 100 is a server computer.

Examples of processing circuitry 102 include one or more microprocessors, digital signal processors (DSPs), application specific integrated circuits (ASICs), field programmable gate arrays (FPGAs), discrete logic, software, hardware, firmware or any combinations thereof. In general, processing circuitry 102 may be implemented as fixed-function circuits, programmable circuits, or a combination thereof. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Processing circuitry 102 may include arithmetic logic units (ALUs), elementary function units (EFUs), digital circuits, analog circuits, and/or programmable cores, formed from programmable circuits. In examples where the operations of processing circuitry 102 are performed using software executed by the programmable circuits, memory 104 may store the object code of the software that processing circuitry 102 receives and executes, or another memory within processing circuitry 102 (not shown) may store such instructions. Examples of the software include software designed for surgical planning.

Memory 104 may be formed by any of a variety of memory devices, such as dynamic random access memory (DRAM), including synchronous DRAM (SDRAM), magnetoresistive RAM (MRAM), resistive RAM (RRAM), or other types of memory devices. Examples of display 110 include a liquid crystal display (LCD), a plasma display, an organic light emitting diode (OLED) display, or another type of display device.

Device 100 may include communication interface 112 that allows device 100 to output data and instructions to and receive data and instructions from visualization device 116 via network 114. For example, after determining a pre-morbid characteristic of the anatomical object, using techniques described in this disclosure, communication interface 112 may output information of the pre-morbid characteristic to visualization device 116 via network 114. A surgeon may then view a graphical representation of the pre-morbid anatomical object with visualization device 116 (e.g., possibly with the pre-morbid anatomical object overlaid on top of image of the injured or diseased anatomical object).

Communication interface 112 may be hardware circuitry that enables device 100 to communicate (e.g., wirelessly or using wires) to other computing systems and devices, such as visualization device 116. Network 114 may include various types of communication networks including one or more wide-area networks, such as the Internet, local area networks, and so on. In some examples, network 114 may include wired and/or wireless communication links.

Visualization device 116 may utilize various visualization techniques to display image content to a surgeon. Visualization device 116 may be a mixed reality (MR) visualization device, virtual reality (VR) visualization device, holographic projector, or other device for presenting extended reality (XR) visualizations. In some examples, visualization device 116 may be a Microsoft HOLOLENS™ headset, available from Microsoft Corporation, of Redmond, Wash., USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses.

Visualization device 1116 may utilize visualization tools that are available to utilize patient image data to generate three-dimensional models of bone contours to facilitate preoperative planning for joint repairs and replacements. These tools allow surgeons to design and/or select surgical guides and implant components that closely match the patient's anatomy. These tools can improve surgical outcomes by customizing a surgical plan for each patient. An example of such a visualization tool for shoulder repairs is the BLUEPRINT™ system available from Wright Medical Technology, Inc. The BLUEPRINT™ system provides the surgeon with two-dimensional planar views of the bone repair region as well as a three-dimensional virtual model of the repair region. The surgeon can use the BLUEPRINT™ system to select, design or modify appropriate implant components, determine how best to position and orient the implant components and how to shape the surface of the bone to receive the components, and design, select or modify surgical guide tool(s) or instruments to carry out the surgical plan. The information generated by the BLUEPRINT™ system is compiled in a preoperative surgical plan for the patient that is stored in a database at an appropriate location (e.g., on a server in a wide area network, a local area network, or a global network) where it can be accessed by the surgeon or other care provider, including before and during the actual surgery.

As illustrated, memory 104 stores data representative of one or more hybrid shape models 106 and data representative of anatomy scans 108. Anatomy scans 108 are, for example, computed tomography (CT) scans of a patient (e.g., as represented by CT scan image data). Anatomy scans 108 may be sufficient to reconstruct a three-dimensional (3D) representation of the anatomy of the patient, such as the scapula and humerus as examples of patient anatomy (e.g., by automated segmentation of the CT image data to yield segmented anatomical objects). Anatomy scans 108 include image data of multiple, related anatomical objects that, when used together, are sufficient to reconstruct a three-dimensional (3D) representation of related anatomical objects of the patient. In some examples, anatomy scans 108 include image data representative of a single scan that includes all of the related anatomical objects. In some examples, anatomy scans 108 include image data representative of a multiple scans that that each primarily include one of the related anatomical objects. One example way of automated segmentation is described in U.S. Pat. No. 8,971,606. There may be various other ways in which to perform automated segmentation, and the techniques are not limited to automated segmentation using techniques described in U.S. Pat. No. 8,971,606. As one example, segmentation of the CT image data to yield segmented objects includes comparisons of voxel intensity in the image data to determine bony anatomy and comparisons to estimated sizes of bony anatomy to determine a segmented object. Moreover, the example techniques may be performed with non-automated segmentation techniques, where a medical professional evaluates the CT image data to segment anatomical objects, or some combination of automation and user input for segmenting anatomical objects.

In one or more examples, anatomy scans 108 may be scans that include one anatomical object that is pathological due to injury or disease (sometimes referred to as “a pathological anatomical object” or a “first anatomical object”) and one or more anatomical objects that are (i) not injured or diseased (sometimes referred to as “non-pathological anatomical object(s)”) and/or (ii) injured or diseased, but provide more complete image data in anatomy scans 108 (sometimes referred to as “fidelitous anatomical object(s),” collectively referred to with “non-pathological anatomical object(s)” as “second anatomical object(s)”). For example, the patient may have an injured shoulder requiring surgery, and for the surgery or possibly as part of the diagnosis, the surgeon may have requested anatomy scans 108 of the humerus, the glenoid, and the scapula of the patient to plan the surgery. A computing device (like device 100 or some other device) may generate segmentations of the patient anatomy so that the surgeon can view anatomical objects and the size, shape, and interconnection of the objects with other anatomy of the patient anatomy needing surgery.

In one or more examples, processing circuitry 102 may utilize image data of anatomy scans 108 to compare (e.g., size, shape, orientation, etc.) against a hybrid statistical shape model (SSM) as a way to determine characteristics of the patient anatomy prior to the patient suffering the injury or disease. In some examples, processing circuitry 102 may initially compare 3D point data of second anatomical object(s) in the image data of anatomy scans 108 to points in the hybrid SSM to determine an intermediate estimated shape of the pathological anatomical object, and then compare 3D point data of non-pathological points of the first anatomical object to determine a final estimated shape of the first anatomical object.

For instance, anatomy scans 108 provide the surgeon with a view of the current characteristics of the first anatomical object and one or more second anatomical objects. To reconstruct the anatomy (i.e., to represent a pre-morbid state), the surgeon may find it beneficial to have a model indicating the characteristics of the first anatomical object prior to the injury or disease. For instance, the model may be a predictor of the first anatomical object of the patient prior to damage or disease. However, it may be likely the patient did not consult with the surgeon until after the injury occurred or disease progressed, and therefore, a model of the patient anatomy prior to the injury or disease (e.g., pre-morbid or native anatomy) may not be available. Using the hybrid SSM as a way to model the pre-morbid anatomy allows the surgeon to determine characteristics of the pre-morbid patient anatomy.

SSMs are a compact tool to represent shape variations among a population of samples (database). For example, a clinician or researcher may generate a database of image scans, such as CT image data, of different people representative of the population as a whole who do not suffer from damaged or diseased glenoid, scapula, humerus, or neighboring bones. The clinician or researcher may determine a mean shape for the patient anatomy from the shapes in the database. A hybrid SSM represent shape variations of multiple, related anatomical objects such that the size and shape of one anatomical object relates to the size and shape of the other anatomical object(s) within the hybrid SSM. In FIG. 1, memory 104 stores information indicative of hybrid shape model 106. As one example, hybrid shape model 106 represents a mean shape for the anatomical objects (e.g., humerus and scapula, etc.) of patient anatomy from the shapes in the database. Other examples of hybrid shape model 106 are possible, such as a mode or median of the shapes in the database. A weighted average is another possible example of hybrid shape model 106. Hybrid shape model 106 may be a surface or volume of points (e.g., a graphical surface or a volume of points that define a 3D model), and the information indicative of mean shape may be coordinate values for vertices of primitives that interconnect to form the surface.

With shape model 106, processing circuitry 102 may represent anatomy shape variations of multiple, related anatomical objects by adding points or values of shape model 106 to a covariance matrix. For example, the SSM can be interpreted as a linear equation:

sᵢ=s′+Σᵢ bᵢ\sqrt{λᵢ}×vᵢ.

In the above equation, s′ is shape model 106 (e.g., point cloud of mean shape as one example, where point cloud defines coordinates of points within shape model 106, such as vertices of primitives that form shape model 106). In the equation, λᵢ is the eigenvalues and vᵢ is the eigenvectors of the covariance matrix respectively (also called modes of variations). The covariance matrix represents the variance in a dataset. The element in the i, j position is the co-variance between the i-th and j-th elements of a dataset array.

SSM stands for constructing the covariance matrix of the database then performing “singular value decomposition” which extracts a matrix of principal vectors (also called eigenvectors) and another diagonal matrix of positive values (called eigenvalues). Eigenvectors (vᵢ in the equation) are new coordinate system bases of the database. Eigenvalues (λᵢ in the equation) represent the variance around the eigenvectors (vᵢ). Together eigenvectors and eigenvalues may reflect the amount of variation around the corresponding axis.

This mathematical equation allows processing circuitry 102 to create an infinite number of instances of sⱼ (e.g., different variations of the hybrid shape model) by simply changing the weights bᵢ of the covariance matrix. For instance, to generate a new shape model, processing circuitry may determine a value of bᵢ, and determine a new value of sᵢ. In the above example, λᵢ and vᵢ and s′ are all known based on the manner in which s′ was generated (e.g., based on the manner in which shape model 106 was generated). By selecting different values of bᵢ, processing circuitry 102 may determine different instances of a shape model (e.g., different sᵢ which are different variations of the shape model 106).

The hybrid shape model(s) may represent what related anatomical objects should look like for a patient. Anatomical objects are related when the characteristics of one of the anatomical objects influence the characteristics of the other anatomical objects. In some examples, anatomical objects are related when the two anatomical objects are connected via a joint. For example, the first anatomical object may connect to the second anatomical object via a joint such that movement of one anatomical object necessitates the movement of the other anatomical object. As described in more detail, processing circuitry 102 may compare points (e.g., in the 3D cloud of points) of the hybrid shape model of the anatomical objects with anatomical points represented in the image data of scans 108. Processing circuitry 102 may generate an intermediate shape model from shape model 106 by comparing points of the hybrid shape model of the anatomical objects to anatomical points of the second anatomical object. Subsequently, in some examples, processing circuitry 102 may generate a final shape model by comparing points of the intermediate shape model to points in the first anatomical object that are not impacted or minimally impacted by the injury or disease (e.g., non-pathological points). Additionally, in some examples, processing circuitry 102 may also compare points of the intermediate shape model to points in the second anatomical object to generate a final shape model that best fits both anatomical objects. Based on the comparison, processing circuitry 102 may determine a pre-morbid characterization of the first anatomical object. The processing circuitry 102 may also determine a characterization of the second anatomical object.

As an example, assume that the surgeon would like to determine the pre-morbid characteristics of the humerus for surgery to repair a humeral head fracture. There is a correlation between the shape of the humerus and scapula. Hybrid shape model 106 may include the mean shapes of the scapula and the humerus. Assume that in the patient the humerus above the surgical neck is pathological (e.g., diseased or damaged).

In accordance with example techniques described in this disclosure, processing circuitry 102 may determine an instance of “s” (“sᵢₙₜₑᵣ”) (e.g., a hybrid shape model of scapula and humerus) that best matches the non-pathological anatomical object of the patient (e.g., the scapula in scans 108). Because the shape model 106 is a hybrid shape model, “sᵢₙₜₑᵣ” includes both the scapula and the humerus. Processing circuitry 102 may then determine, starting with “sᵢₙₜₑᵣ” as “s′,” an instance of “s” (“s_{final}”) that best matches the non-pathological portions of a first anatomical object (e.g., the pathological humerus of scans 108) but may also match the image data for both anatomical objects, rather than just image data for first anatomical object. In some examples, “s_{final}” is the hybrid shape model that best matches the non-pathological or more complete anatomical object (e.g., the second anatomical object) of the patient and the non-pathological portions of a pathological anatomical object (e.g., the first anatomical object). The humerus in the instance of “s_{final}” may be indicative of the pre-morbid characterization of the pathological humerus.

Processing circuitry 102 may perform the following example operations: (1) initially align the segmented anatomical objects from image data of scans 108 to a coordinate system of shape model 106 to generate an initial aligned shape; (2) compensate for errors in the initial alignment due to under and over segmentation, where under- and over-segmentation are due to imperfections in generating scans 108, to generate an aligned shape (also called intermediate aligned shape); (3) perform iterative operations that includes iterative closest point (ICP) operations and elastic registration to determine the instance of sᵢₙₜₑᵣ (e.g., the instance of s that most closely matches the second anatomical object as identified by the segmentation object); and (4) perform iterative operations that includes iterative closest point (ICP) operations and elastic registration to determine the instance of s_{final} (e.g., the instance of s, starting with sᵢₙₜₑᵣ, that most closely matches the first anatomical object as identified by the segmentation object). Example techniques to perform these operations are described in more detail below.

There may be a few technical problems with generating the pre-morbid characterization of the patient anatomy. One issue may be that due to under- or over-segmentation the image data needed from scans 108 is not available or distorted creating a challenge to align the segmented object to hybrid shape model 106. Another issue may be that once there is alignment to shape model 106, registration of shape model 106 to the segmented object may be poor, resulting in poor pre-morbid characterization.

As described above, processing circuitry 102 may align the segmented object (e.g., as segmented using example techniques such as voxel intensity comparisons) to hybrid shape model 106. Alignment refers to changes to the coordinate system of the segmented object so that the segmented object is in same coordinate system as hybrid shape model 106. One example of shape model 106 is a shape model of the anatomy of the scapula and the humerus, where a portion of the humerus the injured or diseased. For instance, hybrid shape model 106 is defined in its own coordinate system and may be different than the patient coordinate system (e.g., coordinate system to define the point cloud of 3D points in scans 108). The patient coordinate system is the coordinate system used to define points within the anatomy of the patient in scans 108 that includes points of the non-pathological anatomical object and non-pathological points and pathological points of the pathological anatomical object. The non-pathological points of the pathological anatomical object refer to the points in scans 108 associated with non-pathological portions of pathological anatomical object, and the pathological points refer to points in scans 108 associated with pathological portions of the pathological anatomical object.

There may be various ways in which to determine pathological and non-pathological points. As one example, the surgeon may review the image data of scans 108 to identify pathological and non-pathological points. As another example, the surgeon may review a graphical representation of the segmented object to identify pathological and non-pathological points. As another example, there may be an assumption that certain anatomical points are rarely injured or diseased and are present in image data of scans 108.

In one or more examples, processing circuitry 102 may determine a patient coordinate system, which is the coordinate system recorded for the CT scan data of scans 108. Hybrid shape model 106 may be defined in its own coordinate system. Processing circuitry 102 may determine the coordinate system of hybrid shape model 106 based on the metadata stored with hybrid shape model 106, where the metadata was generated as part of determining the mean of the shapes in the database. Processing circuitry 102 may determine a transformation matrix based on the patient coordinate system and the coordinate system of shape model 106. The transformation matrix is a way by which processing circuitry 102 transforms the segmented object (e.g., points in the 3D volume of points) into the coordinate system of shape model 106. For example, points in the 3D volume of points of the segmented object may be defined with (x, y, z) coordinate values. The result of the transformation may be coordinates (x′, y′, z′) coordinate values that are aligned to the coordinate system of shape model 106. Processing circuitry 102 calculates the transformation matrix through a close-form equation.

There are multiple ways to determine the transformation matrix. As one example, the coordinate system may be c=(x,y,z,o), where x, y and z are the orthogonal basis 3D vectors and o is the origin (c is a 4×4 homogeneous matrix with the last raw is (0,0,0,1)). The new coordinate system to which the segmented object is to be transformed is C=(X,Y,Z,O). In this example, the transformation matrix is then: T=c\hat{ }−1×C.

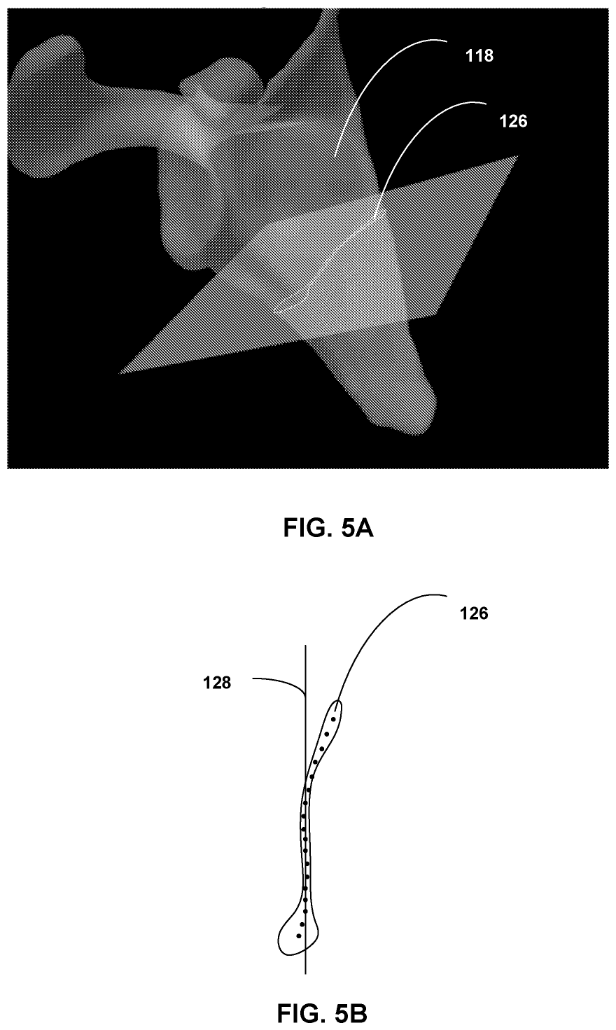

Accordingly, processing circuitry 102 may determine the patient coordinate system. FIGS. 4 to 10 below describe example ways in which processing circuitry 102 determines the patient coordinate system. As described in more detail, even after determining the patient coordinate system, further adjustment to hybrid shape model 106 may be needed to properly align the segmented object (e.g., patient anatomy) to shape model 106. For instance, because of over- or under-segmentation, missing or distorted image data cannot be used to determine the patient coordinate system. Therefore, processing circuity 102 utilizes image data that is available to perform an initial alignment, but then may perform further operations to fully align the segmented object to hybrid shape model 106.

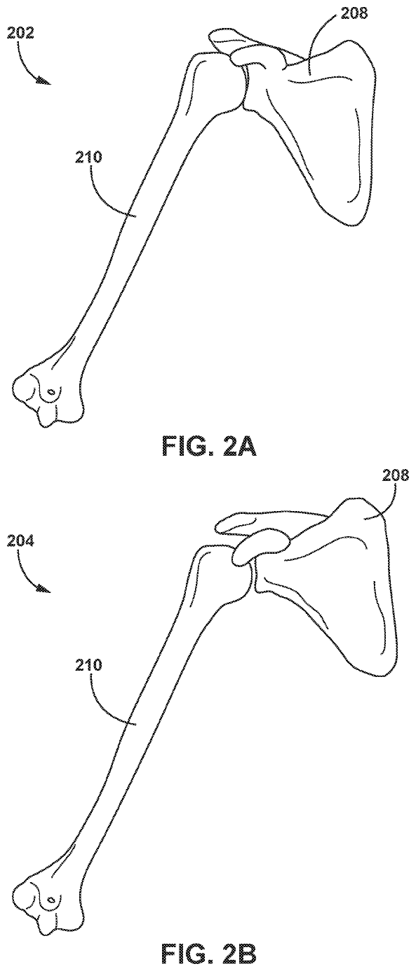

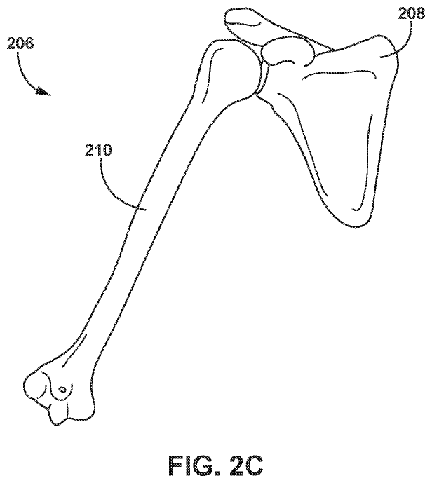

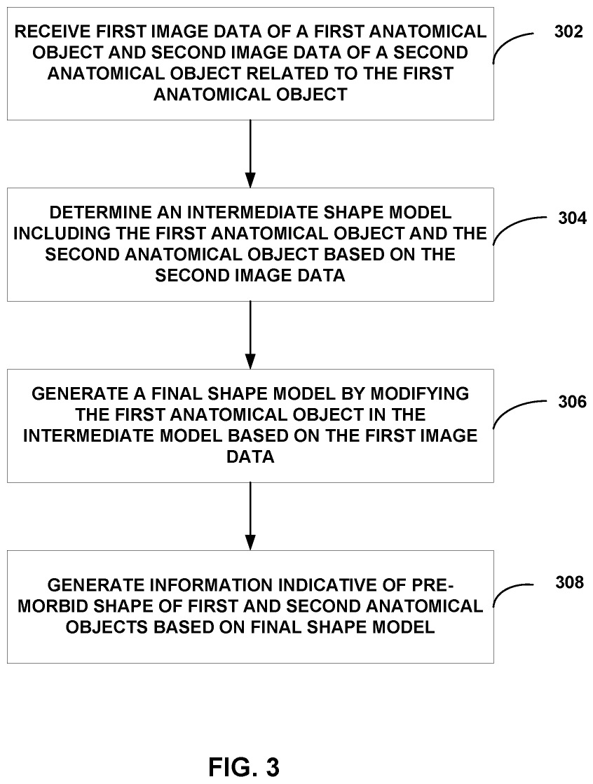

FIGS. 2A, 2B, and 2C illustrate an example transformation of a shape model 202 into an intermediate shape model 204, and then into a final shape model 206. Specially, FIG. 2A illustrates mean shape model 202 that is modified as described herein. Shape model 202 is an example of hybrid shape model 106 of FIG. 1. In the illustrated example of FIG. 2A, shape model 202 includes an average (e.g., mean) shape of a combination of a scapula 208 a humerus 210. When processing circuitry 102 modifies shape model 202 with image data of one anatomical object (e.g., image data of scapula 208), the other anatomical object (e.g., humerus 210) is also changed. FIG. 2B illustrates intermediate shape model 204 formed by modifying mean shape model 202 of FIG. 2A with image data of a first anatomical object. Intermediate shape model 204 is an illustrated example of “sᵢₙₜₑᵣ.” In the illustrated example, shape model 202 is modified using image data of a scapula. In the illustrated example, changes to the scapula 208 also cause changes to humerus 210. FIG. 2C illustrates final shape model 206 formed by modifying intermediate shape model 204 of FIG. 2B with image data of a second anatomical object (e.g., humerus 210) anatomically related to the first anatomical object (e.g., scapula 208). Final shape model 206 is an illustrated example of “s_{final}.” In the illustrated example, intermediate shape model 204 is modified based on image data of humerus 210, and in some examples, image data of scapula 208.

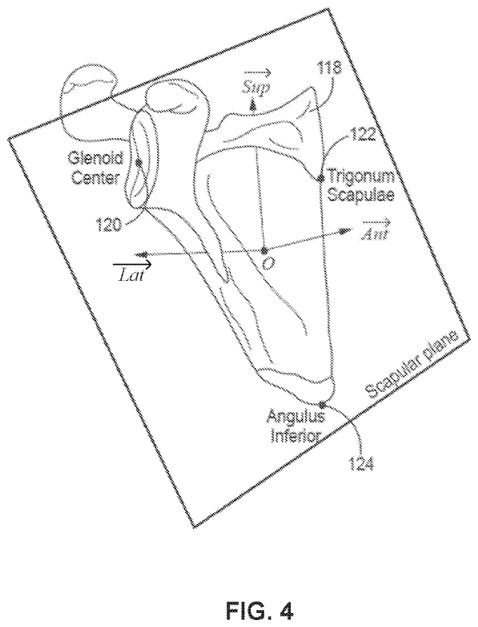

FIG. 3 is a flowchart illustrating an example method of operation in accordance with one or more example techniques described in this disclosure. Initially, processing circuitry 102 receives first image data of a first anatomical object and second image data of a second anatomical object related to the first anatomical object. (302). In some examples, the first image data and the second image data may be obtained from a same image. For example, first image data of a scapula and second image data of a humerus may be obtained from an image containing both the scapula and the humerus. As described below, processing circuitry 102 determines an intermediate shape model (e.g., intermediate shape model 204 of FIG. 2B above) by aligning the hybrid shape model 106 to the second image data of the second anatomical object (304). The hybrid shape model 106 and the intermediate shape model include representations of the first anatomical object and the second anatomical object. When processing circuitry 102 aligns the hybrid shape model 106 to the second image data of the second anatomical object, both representations of the first anatomical object and the second anatomical object of the hybrid shape model 106 may change. Processing circuitry 102 generates a final shape model (e.g., finalshape model 206 of FIG. 2C above) by aligning the intermediate model to the first image data (306). When processing circuitry 102 aligns the intermediate shape model to the first image data of the first anatomical object, the first representation of the first anatomical object of the intermediate shape model changes along with the first representation of the first anatomical object in the intermediate shape model. For instance, the second representation of the second anatomical object of the intermediate shape model also changes as more information is provided by the first image data. As described below, processing circuitry 102 generates information indicative of pre-morbid shapes of the first and second anatomical structures based on the first estimated shape model (308).

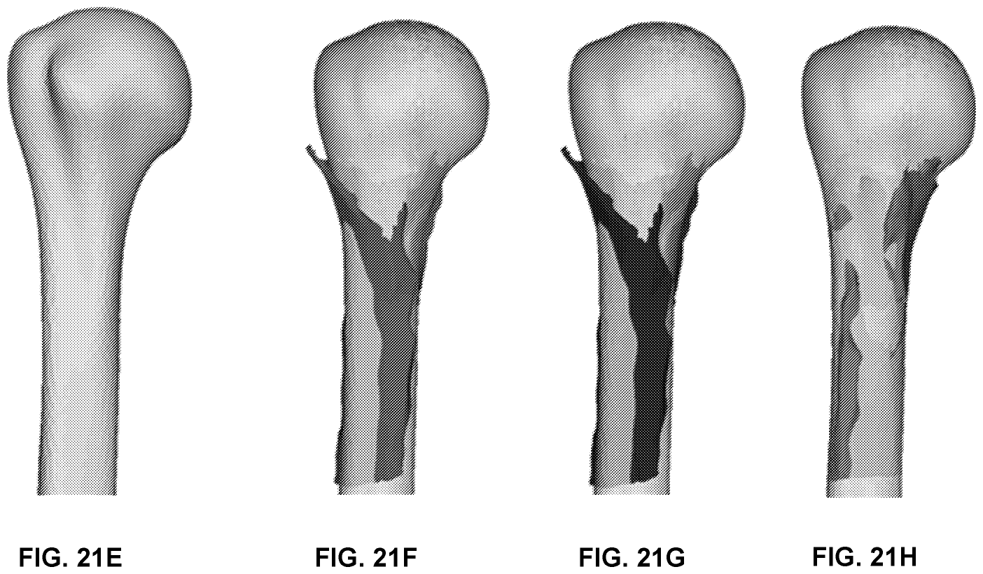

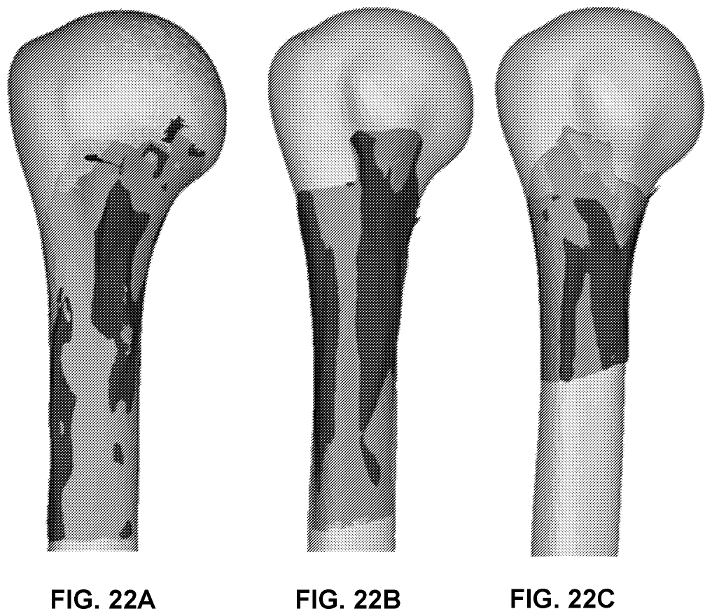

FIGS. 4-17 illustrate examples of determining and aligning a shape model for a scapula. In some examples, when the image data of the scapula includes more data than the humerus, in accordance with the techniques described above, processing circuitry 102 may generate the intermediate model using the images of the scapula. FIGS. 21A-21H and 22A-22C illustrated examples of determining and aligning a shape model for a humerus. While the examples describe aligning and registering segmented image data to a shape model that includes a signal anatomical object, the same techniques may also be used to align and register a hybrid shape model to multiple, related anatomical objects.

In some examples, when the image data of the humerus includes more data than the scapula, in accordance with the techniques described above, processing circuitry 102 may generate the intermediate model using the images of the humerus. However, the techniques in the illustrated example may also be used to determine and align a shape model of any related anatomical object. In such examples, the shape model for the scapula is associated with a shape model of a humerus such that when the shape model of the scapula is determined and aligned, a first prediction of the humerus is also determined and aligned. Subsequently, based on the images of the humerus and the first prediction, the process is repeated to determine and align the shape model of the humerus.

FIG. 4 is an illustration of a scapula having points of interest used to determine coordinate system of a patient. For instance, FIG. 4 illustrates scapula 118 having glenoid center 120, trigonum scapulae 122, and angulus inferior 124. Some techniques utilize glenoid center 120, trigonum scapulae 122, and angulus inferior 124 to determine the patient coordinate system. For instance, glenoid center 120, trigonum scapulae 122, and angulus inferior 126 may be considered as forming a triangle, and processing circuitry 102 may determine a center point (e.g., in 3D) of the triangle as the origin of the patient coordinate system.

However, such techniques may not be available in cases of over- and under-segmentation. As one example, in under-segmentation, scans 108 may be inferiorly and/or superiorly and/or medially truncated and trigonum scapulae 122 and angulus inferior 124 may not be present in the segmented objects extracted from the image data of scans 108. Therefore, processing circuitry 102 may not be able to utilize all three of glenoid center 120, trigonum scapulae 122, and angulus inferior 124 for purposes of determining patient coordinate system.

In one or more examples, where specific landmarks (e.g., glenoid center 120, trigonum scapulae 122, and angulus inferior 124) are not available as segmented anatomical objects in the image data of scans 108, processing circuitry 102 may define the patient coordinate system based on the scapula normal (e.g., a vector normal to scapula 118) and a transverse axis based on the 3D information of image data of scans 108. As described in more detail, processing circuitry 102 may determine the scapula normal based on the normal of the best fit plane to the body of scapula 118. Processing circuitry 102 may determine the transverse axis by fitting a line through the spongious region that lies between the supraspinatus fossa, the spine, and the scapula body. Using the spongious region, processing circuitry 102 may define a Cartesian (e.g., x, y, z) coordinate system (although other coordinate systems are possible). The origin of the coordinate system may be glenoid center 120; although other origins are possible.

In this manner, processing circuitry 102 may determine a coordinate system for the patient based on anatomical objects that are present in the image data of scans 108, even where there is over- or under-segmentation. Based on the coordinate system, processing circuitry 102 may align the segmentation objects to the coordinate system of shape model 106 (e.g., initial SSM) and iteratively deform shape model 106 to register a shape model (e.g., a deformed version of shape model 106) to the patient segmentation object to determine a pre-morbid characterization.

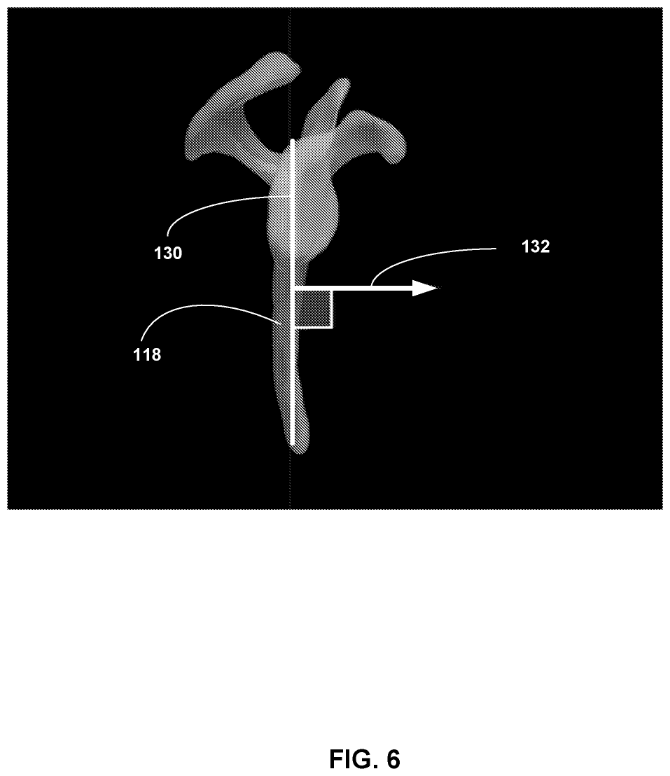

FIGS. 5A and 5B are illustrations of a planar cut through a scapula for determining a patient coordinate system that may not rely upon anatomical objects whose image data is unavailable due to over- or under-segmentation. For instance, unlike example of FIG. 4 that relied upon glenoid center 120, trigonum scapulae 122, and angulus inferior 124, which may not be available due to under- or over-segmentation, the example techniques illustrated with respect to FIGS. 5A and 5B may not rely on anatomical objects that are unavailable due to under- or over-segmentation.

As illustrated in FIG. 5A, one of scans 108 may be a scan of an axial cut through scapula 118, which shows scapula portion 126. FIG. 5B is a top view of scapula portion 126 of FIG. 5A through scapula 118.

FIG. 5B illustrates a plurality of dots, representing an intersecting plane through portion 136, that go through scapula portion 126. In one or more examples, processing circuitry 102 may determine a plane that intersects through scapula portion 126. The intersection of the plane through portion 126 is shown with the dots. For example, FIG. 5B illustrates line 128. Line 128 intersects a majority of the dots in scapula portion 126.

The dots in scapula portion 126 may be the “skeleton” of the surrounding contour. The skeleton is dots through the contour that are each the same distance to their respective nearest boundary. Although the skeleton is described, in some examples, the dots in scapula portion 126 may be center points, as another example.

Processing circuitry 102 may determine the image data that forms portion 126. Processing circuitry 102 may determine dots of portion 126 (e.g., skeleton dots or center dots as two non-limiting examples), as illustrated. Processing circuitry 102 may determine a plane that extends up from line 128 toward the glenoid and extends downward from line 128 toward the angulus inferior.

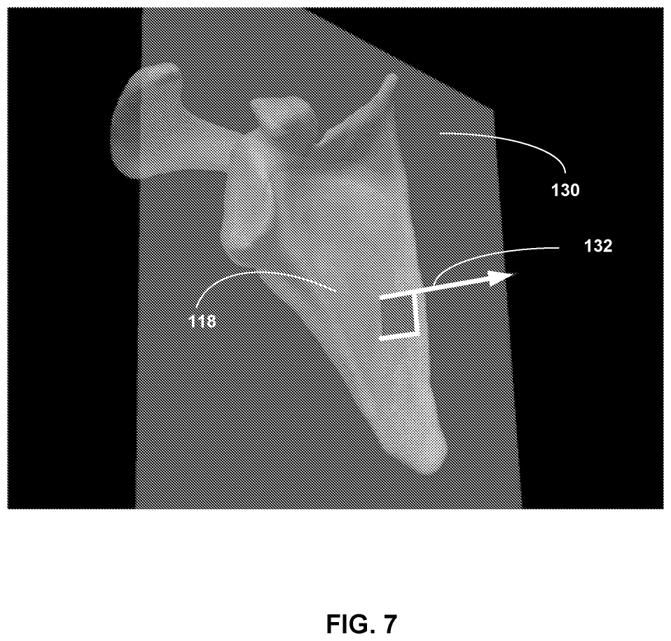

For example, although FIG. 5A illustrates one example axial cut, processing circuitry 102 may determine a plurality of axial cuts, and determine the points through the axial cuts, similar to FIG. 5B. The result may be a 2D points for each axial cut, and processing circuitry 102 may determine a line, similar to like 128, through each of the axial cuts. Processing circuitry 102 may determine a plane that extends through the lines for each axial cut through scapula 118. This plane is through scapula 118 and is illustrated in FIG. 6.

FIG. 6 is a conceptual diagram of a perspective view of a normal line to a plane, as determined with the example illustrated in FIGS. 5A and 5B, through a scapula for determining a coordinate system of a patient. For instance, FIG. 6 illustrates plane 130. Plane 130 intersects line 128 illustrated in FIG. 5B. Plane 130 may be considered as the best fit plane to the body of scapula 118. Processing circuitry 102 may determine vector 132 that is normal to plane 130. In one or more examples, vector 132 may form one axis (e.g., x-axis) of the patient coordinate system. Techniques to determine the other axes is described in more detail below.

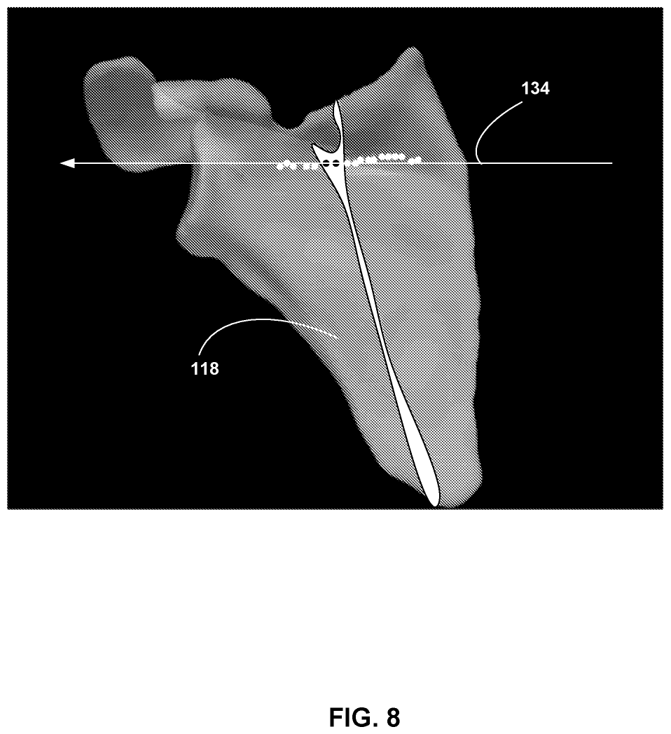

FIG. 7 is a conceptual diagram of another perspective view of the example of FIG. 6 such as another perspective view of a normal line to a plane through a scapula for determining a coordinate system of a patient. FIG. 7 is similar to FIG. 6 but from a front perspective rather than the side perspective of FIG. 6. For example, FIG. 7 illustrates scapula 118 of the patient with the best fit plane 130 and the normal vector 132.

In this manner, processing circuitry 102 may determine a first axis of the patient coordinate system that is normal to scapula 118. Processing circuitry 102 may determine a second axis of the patient coordinate system as an axis that is transverse through scapula 118.

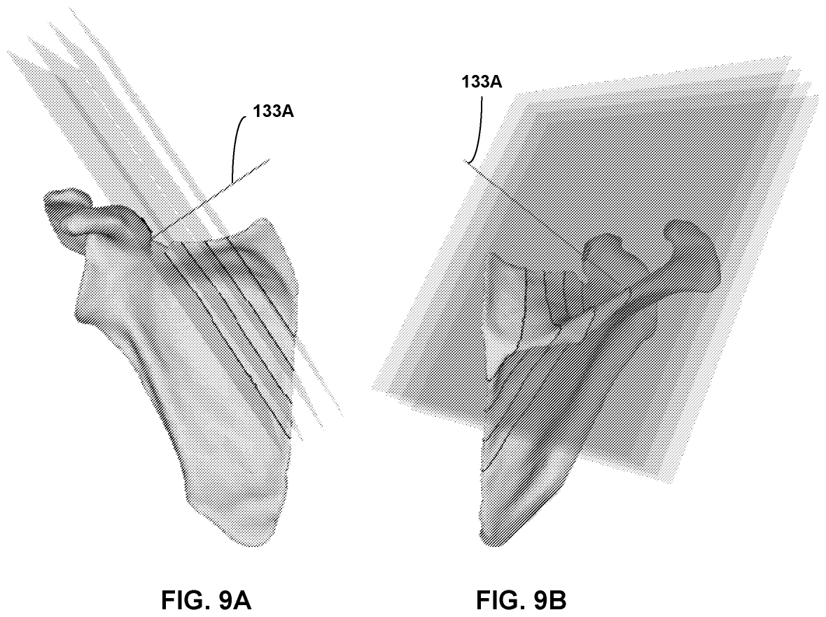

FIG. 8 is a conceptual diagram illustrating a transverse axis through a scapula for determining a coordinate system of a patient. For instance, FIG. 8 illustrates example to determine an axis, in addition to the one determined in FIGS. 6 and 7, for aligning shape model 106 to coordinate system of the image data of scans 108.

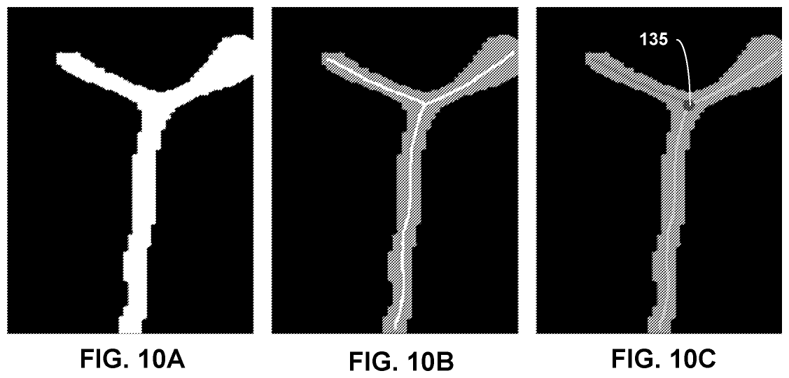

FIG. 8 illustrates transverse axis 134. Processing circuitry 102 may determine transverse axis 134 as a line that fits through the spongious region that lies between the supraspinatus fossa, the spine, and the scapula body. The example techniques to determine transverse axis 134 is described with respect to FIGS. 9A, 9B, FIGS. 10A-10C, and FIGS. 11A and 11B, and. Processing circuitry 102 may determine plurality of sagittal cuts through the scapula based on the image data of scan 108 as illustrated in FIGS. 9A and 9B. For example, FIGS. 9A and 9B are different perspectives of sagittal cuts through scapula based on axis 133A. Processing circuitry 102 may utilize axis 133A based on an approximation of the spongious region that lies between supraspinatus fossa, the spine, and the scapula body, and may be pre-programmed with the estimated axis.

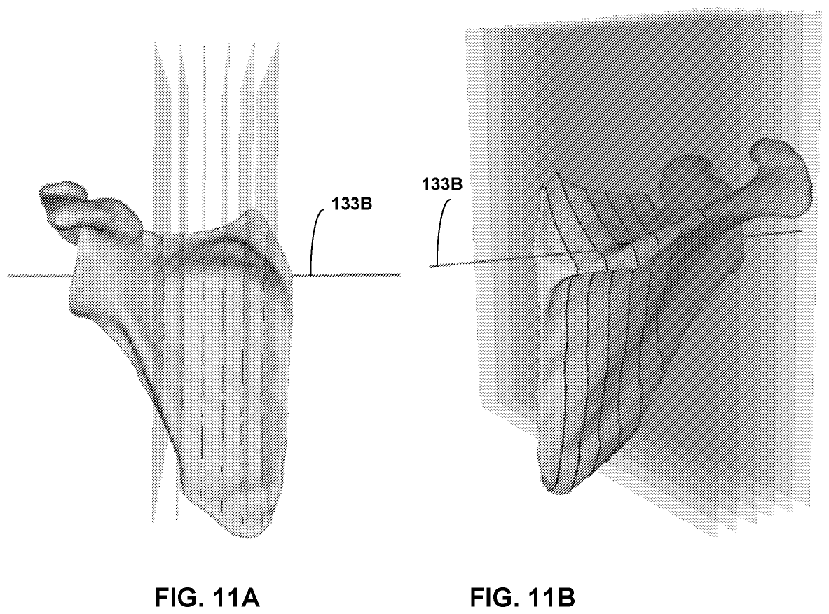

FIGS. 10A-10C illustrate the result of one of the sagittal cuts of the FIGS. 9A and 9B. For instance, the sagittal cuts from a “Y” shape, as illustrated in FIG. 10A. Processing circuitry 102 may determine a skeleton line through the Y shape, as illustrated in FIG. 10B. For instance, processing circuitry 102 may determine dots that are equidistant to nearest border through the Y shape and interconnect the dots to form the line through Y shape as shown in FIG. 10B. Again, rather than skeleton, other points may be used like center points. Processing circuitry 102 may determine an intersection of the lines through the skeleton, as illustrated by intersection point 135. For instance, intersection point 135 may be common to all of the lines that together form the Y shape.

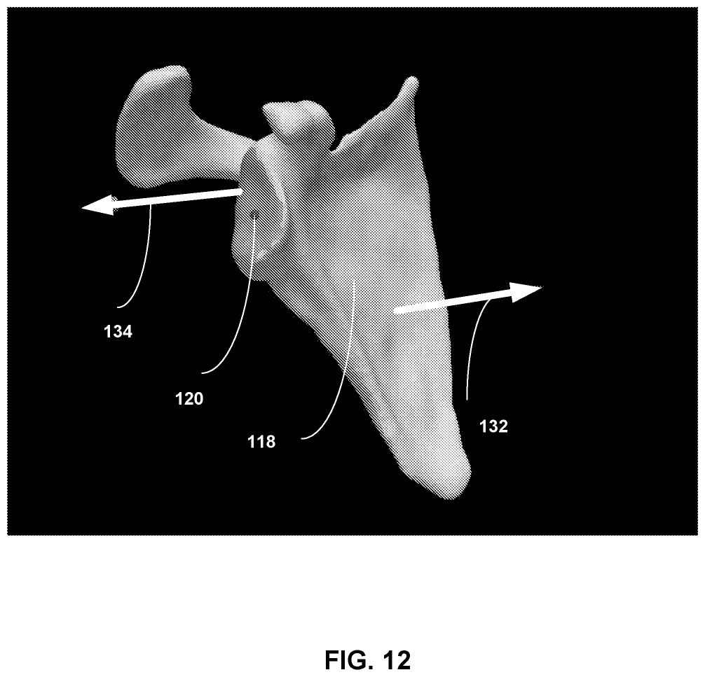

Processing circuitry 102 may repeat these operations for each of the Y shapes in each of the sagittal cuts, and determine respective intersection points, like intersection point 135. Processing circuitry 102 may determine a line that that intersects the plurality of the respective intersection points to determine an initial transverse axis 133B illustrated in FIGS. 11A and 11B.

Processing circuitry 102 may determine sagittal cuts through the scapula using the initial transverse axis 133B. For instance, FIGS. 11A and 11B are different perspectives of sagittal cuts through scapula based on axis 133B. Processing circuitry 102 may repeat the operations described with respect to FIGS. 10A-10C to determine a plurality of intersection points for the sagittal cuts shown in FIGS. 11A and 11B. For example, similar to FIGS. 10A-10C, based on the sagittal cuts using axis 133B, each sagittal cut may form Y shapes, similar to FIG. 10A. Processing circuitry 102 may determine a skeleton line through the Y shapes, similar to FIG. 10B, and determine intersection points similar to FIG. 10C. Processing circuitry 102 may repeat these operations for each of the Y shapes in each of the sagittal cuts, and determine respective intersection points, similar to the description above for FIGS. 10A-10C. Processing circuitry 102 may determine a line that that intersects the plurality of the respective intersection points to determine transverse axis 134 illustrated in FIG. 8.

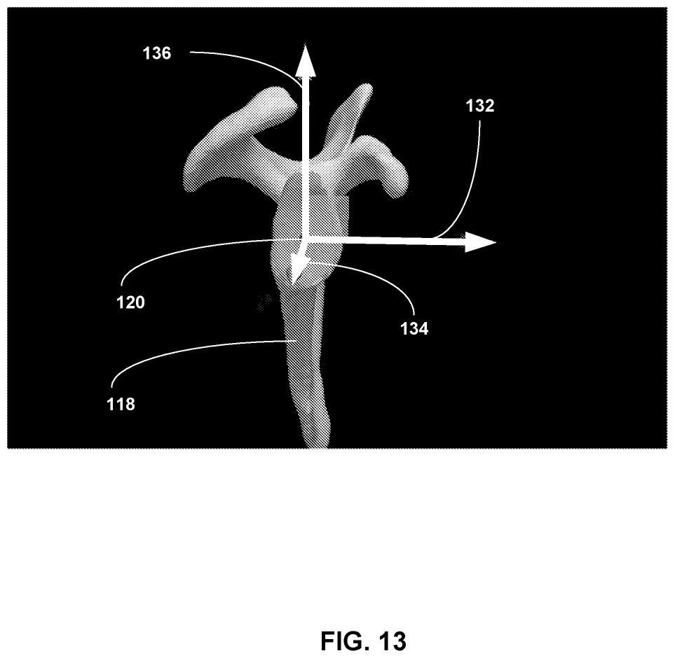

FIG. 12 is a conceptual diagram illustrating a transverse axis of FIG. 8 through a scapula and a normal line of FIGS. 6 and 7 to a plane through the scapula for determining a coordinate system of a patient. For example, FIG. 12 illustrates scapula 118 and glenoid center 120. FIG. 12 also illustrates vector 132 that is normal to plane 130 and illustrates transverse axis 134.

For example, as described above, an initial step for pre-morbid characterization is to determine a coordinate axis with which the segmented objects are aligned to shape model 106. With the example techniques described above, processing circuitry 102 may determine the x-axis (e.g., vector 132) and the z-axis (e.g., transverse axis 134). Processing circuitry 102 may further determine the y-axis using the below techniques. Once process circuitry 102 determines the coordinate system to represent and define location of anatomical objects determined from the segmentation of the image data of scans 108, processing circuitry 102 may be able to align the segmented object to the coordinate system of shape model 106.

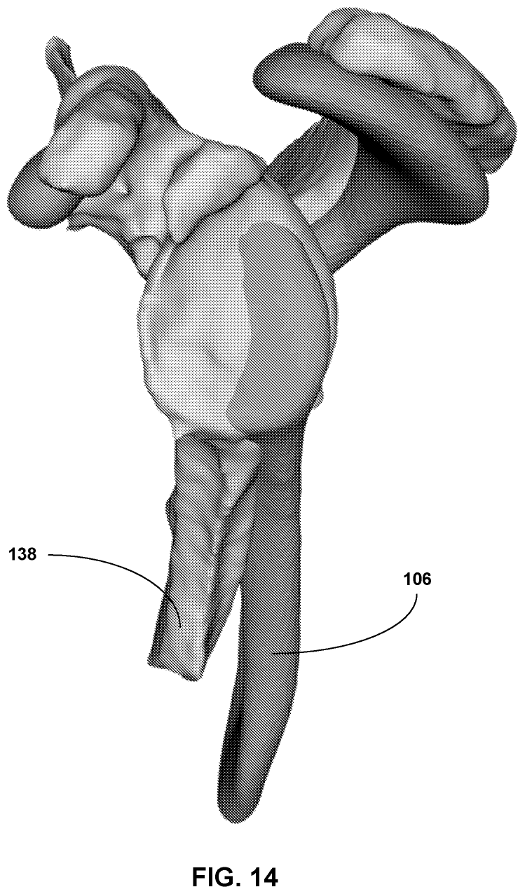

FIG. 13 is a conceptual diagram illustrating movement (or extension) of a transverse axis through a scapula and a normal line to a plane through the scapula to a central location of a glenoid for determining a coordinate system of a patient. For instance, FIG. 10 illustrates the patient coordinate system centered around glenoid center 120. As illustrated, vector 132, as determined above, forms the x-axis of the patient coordinate system, transverse axis 134 forms the z-axis of the patient coordinate system, and axis 136 forms the y-axis of the patient coordinate system.

Processing circuitry 102 may determine a glenoid center based on a plurality of 2D and 3D operations. For example, processing circuitry 102 may determine a barycenter of the glenoid cavity and project the barycenter back to the glenoid cavity surface to determine glenoid center 120.

Processing circuitry 102 may then determine y-axis 136 based on x-axis 132, and z-axis 134. For example, processing circuitry 102 may determine y′=z*x, where * is the vector product. The y′ is perpendicular to the plane defined by the x-axis 132 and the z-axis 134, but x-axis 132 and z-axis 134 need not necessarily be perfectly orthogonal. Accordingly, processing circuitry 102 may replace z-axis 134 by Z=x*y′ or x-axis 132 by X=y′*z to have an orthogonal system (x, y′, Z) or (X, y′, z). In some examples, processing circuitry 102 may utilize Z=x*y′ because the computation of x-axis 132 (e.g., the scapula normal) is more robust than the computation of z-axis 134 (e.g., transverse axis).

In this manner, processing circuitry 102 may determine the patient coordinate system. In some examples, such as those described above, processing circuitry 102 may determine the patient coordinate system without relying upon specific landmarks such as trigonum scapulae 122 and angulus inferior 124 that may not be present in segmentation of anatomical objects from the image data of scans 108 due to over or under segmentation. In particular, the example technique determine x, y, and z-axis without relying on specific anatomical objects, but rather, determine the x, y, and z-axis based on scapula 118 itself, which should be present in the image data of scans 108, whereas trigonum scapulae 122 and angulus inferior 124 may not be present in the image data of scans 108 due to under- or over-segmentation.

After determining the patient coordinate system, processing circuitry 102 may determine the transformation matrix to align the patient coordinate system to shape model 106. As described above, one example way to determine the transformation matrix is using the coordinate system where c=(x,y,z,o), and where x, y and z are the orthogonal basis 3D vectors and o is the origin (c is a 4×4 homogeneous matrix with the last raw is (0,0,0,1)). The new coordinate system to which the segmented object is to be transformed is C=(X,Y,Z,O). In this example, the transformation matrix is then: T=c\hat{ }−1×C

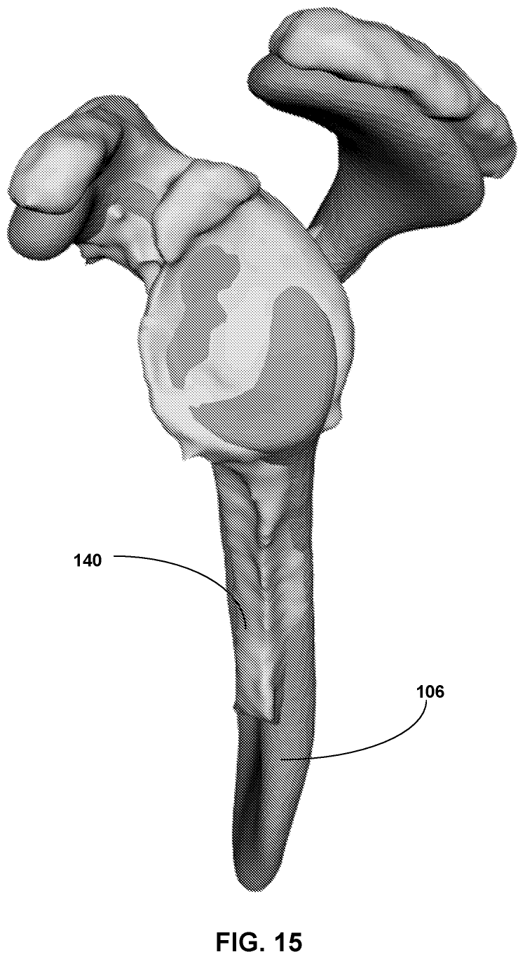

Processing circuitry 102 may multiply the coordinates that define the segmentation object (e.g., where the coordinates are determined from the axes utilizing techniques described above) based on the image data from scans 108 with the transformation matrix to align the segmentation objects to shape model 106 (e.g., the SSM retrieved from memory 104). The result of this alignment may be an initial shape. For example, FIG. 12 illustrates initial aligned shape 138 that is aligned with shape model 106. Initial aligned shape 138 is shown as superimposed on shape model 106, in FIG. 14. Initial aligned shape 138 is generated based on the image data of scans 108 where certain portions of the anatomy may be missing (e.g., due to under-segmentation) or distorted (e.g., due to over-segmentation). For instance, initial aligned shape 138 may be generally positioned such that it has the same orientation on the x-, y-, and z-axis as shape model 106.

However, following transformation, initial aligned shape 138 may not be as closely aligned with shape model 106. In case the scapula is over-truncated inferiorly and/or medially (which may not be known but is the case in initial shape 138), there could be computational errors in determining the scapula body normal (e.g., vector 132) and the transverse axis 134. These computational errors may lead to misalignment between the shape model 106 and the patient anatomy (e.g., scapula 118), and therefore, initial aligned shape 138 may not be as closely aligned with shape model 106 as desirable. For example, as can be seen from FIG. 14, initial aligned shape 138 is rotated along z-axis (e.g., transverse axis) 134.

In one or more examples, because it may be unknown whether there is misalignment from over-truncated scapular 118, processing circuitry 102 may modify parameters of initial shape 138 to generate an aligned shape. The aligned shape is substantially proximal (e.g., in location, size, and orientation) to shape model 106. As one example, to modify parameters, processing circuitry 102 may iteratively adjust coordinates of initial aligned shape 138 so that the initial aligned shape model rotates along the z-axis (e.g., the points of initial aligned shape 138 rotate along the z-axis). At each adjustment, processing circuitry 102 may determine a distance between the initial aligned shape 138 and shape model 106 (e.g., points in the initial aligned shape and points represented in shape model 106).

For instance, processing circuitry 102 may determine the distances between points on initial aligned shape 138 (e.g., such as points of acromion and the coracoid) and corresponding points on shape model 106 (e.g., acromion and coracoid on shape model 106). The corresponding points refer to points in the initial aligned shape 138 and points in shape model 106 that identify the same patient anatomy. Processing circuitry 102 may keep rotating the initial aligned shape 138 until the distance between the initial aligned shape 138 and shape model 106 about the z-axis 134 satisfies a threshold (e.g., less than threshold including where distance is minimized). Then, processing circuitry 102 may rotate the initial aligned shape 138 along the y-axis 138 until the difference in points between initial aligned shape 138 and corresponding points in shape model 106 about the y-axis satisfies a threshold (e.g., less than threshold including where distance is minimized). Processing circuitry 102 may rotate the initial aligned shape model about the x-axis 132 until the difference in points between initial aligned shape 138 and corresponding points in shape model 106 along the x-axis 132 satisfies a threshold (e.g., less than threshold including where distance is minimized).

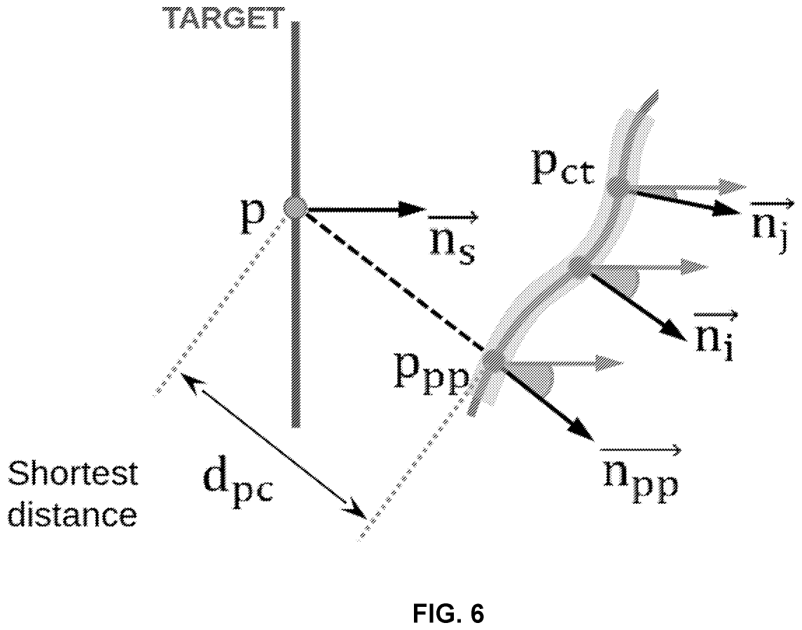

As one example, processing circuitry 102 may modify the parameters of the initial aligned shape 138 so that the initial aligned shape 138 rotates 5-degrees along an axis (e.g., a first instance of the initial aligned shape model) within a search range of [−45-degrees, 45-degrees] or [−27 degrees, 27 degrees]. Processing circuitry 102 may determine the distance between points of the first instance of the initial aligned shape 138 and corresponding points of shape model 106. Assuming the distance is not at a minimum or less than threshold, next, processing circuitry 102 may modify the parameters of the initial aligned shape 138 so that the initial aligned shape rotates 10-degrees along the axis (e.g., a second instance of the initial aligned shape 138) and determine the distance (e.g., between points of the second instance of the initial aligned shape 138 and corresponding points of shape model 106). Processing circuitry 102 may repeat these operations about each of the axes for each instance of the initial aligned shape 138. Processing circuitry 102 may keep repeating these operations until processing circuitry 102 determines an instance of the initial aligned shape 138 that resulted in a distance less than a threshold (such as the minimum distance). The instance of the initial aligned shape 138 that resulted in the distance being less than threshold (including example of minimum distance) among the various iterative instances is selected as aligned shape 140, illustrated in FIG. 15.

The result of these operations may be the aligned shape (e.g., a model that has been rotated along the x, y, and z-axes). For example, FIG. 15 illustrates aligned shape 140 that is aligned to shape model 106. Aligned shape 140 is shown as superimposed on shape model 106, in FIG. 15. Aligned shape 140 (e.g., simply aligned shape 140) provides a better alignment to shape model 106 as compared to initial aligned shape 138. For instance, as shown in FIG. 15, aligned shape 140 is better aligned to shape model 106 than initial aligned shape 138 to shape model 106, as shown in FIG. 14.

For instance, initial aligned shape 138 and shape model 106 may be generally aligned. However, there may be some tilting or misorientation between the initial aligned shape 138 and shape model 106 due to the over or under segmentation of the image data of anatomical objects in scans 108. To address this, processing circuitry 102 may separately rotate initial aligned shape 138 about each axis (x, y, z) until the distance between initial aligned shape 138 and shape model 106 satisfies a threshold (e.g., minimized). Accordingly, by iteratively rotating initial aligned shape 138, processing circuitry 102 may generate aligned shape 140 (also called intermediate aligned shape 140). Because the distance between points on aligned shape 140 and corresponding points of shape model 106 may be minimized, aligned shape 140 (e.g., intermediate aligned shape 140) is substantially in the coordinate system of shape model 106.

Aligned shape 140 may be referred to as an intermediate aligned shape because in some examples, shape model 106 is deformed to the shape of aligned shape 140 to generate the pre-morbid characteristics of the patient anatomy. Processing circuitry 102 may utilize techniques described below to deform shape model 106 to register shape model 106 to aligned shape 140. However, in some examples, processing circuitry 102 may utilize some other techniques to deform shape model 106 to register to aligned shape 140. Also, for the example techniques to register shape model 106 to aligned shape 140, it may be possible that such techniques are preformed where aligned shape 140 is generated using techniques other than those described above.

As described above, processing circuitry 102 may be configured to register shape model 106 to aligned shape 140. Registering shape model 106 to aligned shape 140 may refer to iteratively deforming shape model 106 until a cost function value is below a threshold (e.g., minimized). The registering algorithm is a global loop that includes two inner iterative loops referred to as iterative closest point (ICP) loop and elastic registration (ER) loop. There may be other example ways in which to perform the registering algorithm, and the use of two loops within a global loop is just one example way in which to perform the registering algorithm. For instance, in some examples, it may be possible to bypass the ICP loop and only perform the ER loop. In some examples, it maybe possible to perform only the ICP loop and bypass the ER loop.

In the ICP loop for the first iteration of the global loop, the initial input is aligned shape 140 and shape model 106. For the ICP loop in the first iteration of the global loop, processing circuitry 102 iteratively modifies aligned shape 140 based on a comparison of aligned shape 140 to shape model 106 to generate a first order shape. For example, for each iteration through the ICP loop, processing circuitry 102 determines a cost value, and the modified aligned shape that results in the cost value less than a threshold (e.g., minimized) is the output of the ICP loop and is referred to a first order shape. An example of the ICP algorithm is described in more detail below.

The first order shape is an input into the ER loop for the first iteration of the global loop. Another input into the ER loop, for the first iteration of the global loop, is shape model 106. For the ER loop in the first iteration of the global loop, processing circuitry 102 may deform shape model 106 to generate a plurality of estimated shape models (e.g., one for each time through the ER loop). For each loop of the ER loop, processing circuitry 102 may determine a total cost value, and determine which iteration of the ER loop utilized the estimated shape model having the total cost value that satisfies the threshold (e.g., less than threshold including where minimized). The result of the ER loop (e.g., the estimated shape model having the total cost value that satisfies the threshold) is a first order registered shape model. This completes one iteration of the global loop. The total cost value, for the first iteration of the global loop, may be based on distances and orientation between the estimated shape models and the first order shape, constraints on medialization of anatomy, and parameter weighting.

For example, assume that S1 is the first order shape model that processing circuitry 102 is to determine using the ER loop in the first iteration of the global loop. For the ER loop, processing circuitry 102 may generate S11, S12, S13, and so forth, where each one of S11, S12, S13, and so forth is a deformed version of shape model 106 and S11, S12, S13, and so forth are each an example of an estimated shape model. For each of one of S11, S12, S13, and so forth, processing circuitry 102 may determine a total cost value (e.g., S11_total cost value, S12_total cost value, S13_total cost value, and so forth). Processing circuitry 102 may determine which of the total cost values is the less than a threshold (e.g., minimized). The estimated shape model (e.g., one of S11, S12, S13, and so forth) having the total cost value that is below a threshold (e.g., minimized) is S1 (e.g., the first order registered shape model). After this, the first iteration of the global loop is complete.

For the second iteration of the global loop, processing circuitry 102 performs the operations of the ICP loop. In the second iteration of the global loop, for the ICP loop, one input is the first order shape model, generated from the ER loop, and the other input is the first order shape, generated by the previous ICP loop. In the second iteration of the global loop, for the ICP loop, processing circuitry 102 iteratively modifies the first order shape based on a comparison of the first order shape to the first order shape model. For instance, for each iteration through the ICP loop, processing circuitry 102 determines a cost value, and the modified first order shape that results in the cost value less than a threshold (e.g., minimized) is the output of the ICP loop and is referred to a second order shape.

The second order shape is an input into the ER loop for the second iteration of the global loop. Another input into the ER loop, for the second iteration of the global loop, is shape model 106 and/or the first order shape model. Where shape model 106 is used, the values of bᵢ may range within [−b+b]. Where the first order shape model is used, the values of bᵢ may range within [−b+b_prev b+b_prev], where b_prev is the value found in the previous iteration (e.g., used to determine the first order shape model).

For the ER loop in the second iteration of the global loop, processing circuitry 102 may deform shape model 106 and/or the first order shape model to generate a plurality of estimated shape models (e.g., one for each iteration of the ER loop). For each iteration of the ER loop, processing circuitry 102 may determine a total cost value, and determine which iteration of the ER loop utilized the estimated shape model having the total cost value that satisfies the threshold (e.g., minimized). The result of the ER loop, in the second iteration of the global loop, (e.g., the estimated shape model having the total cost value that satisfies the threshold) is a second order shape model. This completes a second iteration of the global loop. The total cost value, for the second iteration of the global loop, may be based on distances and orientation between the estimated shape models and the second order shape, constraints on medialization of anatomy, and parameter weighting.

For example, assume that S2 is the second order shape model that processing circuitry 102 is to determine using the ER loop in the second iteration of the global loop. For the ER loop, processing circuitry 102 may generate S21, S22, S23, and so forth, where each one of S21, S22, S23, and so forth is a deformed version of shape model 106 and/or the first shape model and S21, S22, S23, and so forth are each an example of an estimated shape model. For each of one of S21, S22, S23, and so forth, processing circuitry 102 may determine a total cost value (e.g., S21_total cost value, S22_total cost value, S23_total cost value, and so forth). Processing circuitry 102 may determine which of the total cost values is the less than a threshold (e.g., minimized). The estimated shape model (e.g., one of S21, S22, S23, and so forth) having the total cost value that is below a threshold (e.g., minimized) is S2 (e.g., the second order registered shape model). After this, the second iteration of the global loop is complete.

In the above example, processing circuitry 102 generated S11, S12, S13, and so forth (e.g., first set of estimated shape models) for the first iteration of the global loop and generated S21, S22, S23, and so forth (e.g., second set of estimated shape models) for the second iteration of the global loop. In some examples, the number of estimated shape models in the first and second set of estimated shape models may be the same. In some examples, the number of estimated shape models in the first and second set of estimated shape models may be different (e.g., 6 estimated shape models in the first set of estimated shape models and 14 estimated shape models in the second set of estimated shape models). One reason for having different numbers of estimated shape models in different iterations of the global loop is that by increasing the number of estimated shape models in subsequent iterations of the global loop, it may be possible to more accurately determine a global minimum as compared to if the same or fewer number of estimated shape models is used. That is, processing circuitry 102 may gradually increase the number of unknowns (e.g., modes and scale factors) used to generate the estimated shape models through multiple ER loops.

This process keeps repeating until the total cost value is below a threshold (e.g., minimized). The registered shape model (e.g., N^{th} order registered shape model) that provides the total cost value below a threshold (e.g., minimized) provides a pre-morbid characterization of the patient anatomy. Some additional processing may be needed to bring back the pre-morbid characterization into the patient coordinate system.

As described above, for determining the pre-morbid characterization, there is a global loop that includes an ICP loop and an ER loop. The following describes an example of the ICP loop. In the ICP loop, there is a source point clout and a target point cloud. The target point cloud remains fixed and processing circuitry 102 modifies (e.g., transforms) the source point cloud such that the transformed point cloud matches the target point cloud. A difference between the transformed point cloud and the target point cloud indicates how well of a match the transformed point cloud is to the source cloud. In one or more examples, processing circuitry 102 keeps transforming the source point cloud until the difference is below a threshold. For instance, processing circuitry 102 keeps transforming the source point cloud until the difference is minimized.

In one or more examples, in the first iteration of the global loop, for the ICP loop, the target point cloud is shape model 106 and the source point cloud is aligned shape 140. Processing circuitry 102 may determine distances between points on aligned shape 140 and corresponding points on shape model 106. The points on aligned shape 140 that are used may be points that are known not be pathological (e.g., medial glenoid vault, the acromion, and the coracoid, as a few non-limiting examples). Other examples include various other points on the scapula that are present in the image data of scans 108. Processing circuitry 102 may find points for the same anatomy on shape model 106.

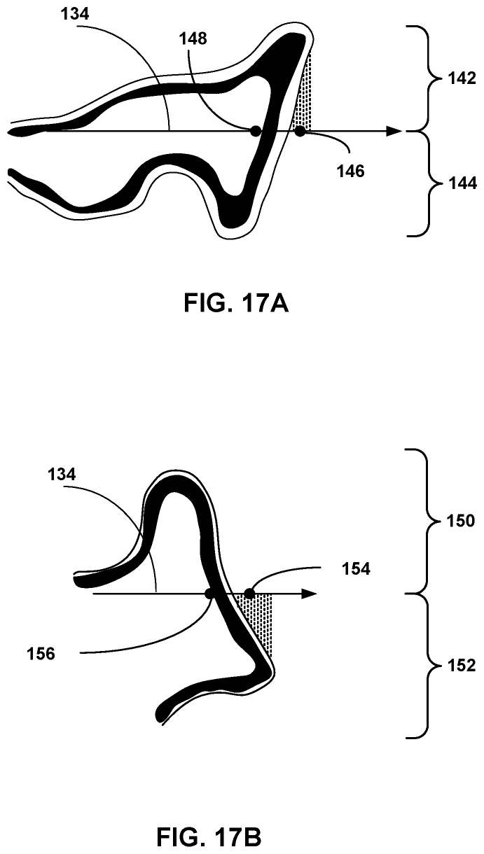

FIG. 16 is a conceptual diagram illustrating an example for determining difference values for ICP algorithm. For example, processing circuitry 102 may determine a point (p) on target point cloud (e.g., shape model 106). Processing circuitry 102 may then determine closest point to point p on aligned shape 140, which in the example of FIG. 16 is point pₚₚ. Processing circuitry 102 may determine a set number of points (e.g., 10 points) that are proximate to point pₚₚ on aligned shape 140 such as p_{ct}, illustrated in FIG. 16.

For point p on shape model 106 and each of these points (e.g., closest point and proximate points on aligned shape 140), processing circuitry 102 may determine a normal vector. For example, vector ns is the normal vector for point p, vector npp is the normal vector for point pₚₚ, and vector nj is the normal vector for point p_{ct}. One way to determine the normal vectors is based on vectors orthogonal to a tangential plane to the point. Another way to compute a point's normal is to calculate the normal of each triangle that shares that point and then attribute the mean normal. In order to overcome local noise, normals are smoothed by computing the mean normal of points within a specific vicinity.

Processing circuitry 102 may determine an orientation and distance difference between point p on shape model 106 and points on aligned shape 140 based on the following equation:

Difference=norm(pₛ−pₜ)²+w*norm(nₛ−nₜ)².

In the above equation, pₛ is a source point (e.g., point p on shape model 106), pt is point on aligned shape 140 (e.g., closest point and proximate points such as point pₚₚ or p_{ct}), ns is the normal vector of point ps (e.g., ns as shown in FIG. 16), nt is the normal vector of point on aligned shape 140 (e.g., npp and nj), and w is a pre-programmed weighting factor. Typically, w equals 1 to give equal weights between distance and orientation. In some examples, high/low curvature regions may require higher/lower values of ‘w’. The value of ‘w’ may be defined empirically.

Processing circuitry 102 may determine which of the difference values resulted in the smallest different value. For example, assume that a first difference value is based on p, ns, pₚₚ, and nₚₚ and a second difference value is based on p, ns, p_{ct}, and nⱼ. In this example, the second difference value may be smaller than the first difference value. Processing circuitry 102 may determine p_{ct} on aligned shape 140 as a corresponding point to point p on shape model 106.

Although FIG. 16 illustrates one point p on shape model 106, there may be plurality (e.g., N number) of points on shape model 106, and processing circuity 102 may perform operations similar to those described above to identify N corresponding points on aligned shape 140 for each of the N points on shape model 106. Accordingly, there may be N difference values. Processing circuitry 102 may sum together the N difference values and divide the result by N. If the resulting value is greater than a threshold (e.g., including examples where the resulting value is not minimized), processing circuitry 102 may continue with the ICP loop.

With the N points on shape model 106 and the N points on aligned shape, processing circuitry 102 may determine a rotation matrix, R, and a translation vector, t. Based on the rotation matrix and the translation vector, processing circuitry 102 may rotate and translate the points on aligned shape 140 (e.g., −R multiplied by (points on aligned shape) plus translation vector). The result is a first intermediate first order shape. One example way of generating the rotation matrix R and the translation vector t is described in Berthold K. P. Horn (1987), “Closed-form solution of absolute orientation using unit quaternions,” https://pdfs.semanticscholar.org/3120/a0e44d325c477397afcf94ea7f285a29684a.pdf.

This may conclude one instance of the ICP loop. Processing circuitry 102 may then repeat these operations where processing circuit 102 uses the first intermediate first order shape, in place of aligned shape 140, and shape model 106. Processing circuitry 102 may determine N difference values for each of the N points on shape model 106 and the N points on the intermediate first order shape. Processing circuitry 102 may sum together the N difference values and divide by N. If the resulting value is greater a threshold (or not minimized), processing circuitry 102 may determine the rotation matrix and the translation vector and determine a second intermediate first order shape. This may conclude a second iteration through the ICP loop.

Processing circuitry 102 may keep repeating these operations until processing circuitry 102 determines a value resulting from the sum of N difference values between the N points on shape model 106 and the N points on the Xth intermediate first order shape being divided by N where the value satisfies a threshold (such as when the value is minimized). In this case, the ICP loop is concluded and processing circuitry 102 determines that the Xth intermediate first order shape is the first order shape.

The first order shape becomes an input into the elastic registration (ER) loop. Another input in the ER loop is shape model 106. In the ER loop, processing circuitry 102 may determine a plurality of estimated shape models based on shape model 106. For instance, processing circuitry 102 may determine a new shape model (sᵢ) based on the following equation:

sᵢ=s′+Σᵢ bᵢ\sqrt{λ₁}×vᵢ.

In the above equation, s′ is shape model 106 (e.g., point cloud of mean shape as one example, where point cloud defines coordinates of points within shape model 106, such as vertices of primitives that form shape model 106). In the equation, λᵢ is the eigenvalues and vᵢ is the eigenvectors of the covariance matrix respectively (also called modes of variations). The covariance matrix represents the variance in a dataset. The element in the i, j position is the co-variance between the i-th and j-th elements of a dataset array.

Processing circuitry 102 may determine the plurality of estimated shape models by selecting different values of bᵢ. In the above, bᵢ is a scaling factor to scale the eigenvalues or eigenvectors. The eigenvalue (λᵢ) the eigenvector (vᵢ) are known from the generation of shape model 106 (e.g., s′). For ease of illustration, assume that processing circuitry 102 determined 10 estimated shape models based on 10 selected (e.g., randomly or pre-programmed) values of bi. There may be more or fewer than 10 estimated shape models.

In the ER loop, processing circuitry 102 may perform the following operations on the estimated shape models. As part of the ER loop, processing circuitry 102 may determine which of the estimated shape models produces a cost function value that is below a threshold (e.g., minimized). The cost function value may be based on three sub-cost function values, although more or fewer than the three sub-cost function values are possible. For example, assume that the first sub-cost function value is Cf1, the second sub-cost function value is Cf2, and the third sub-cost function value is Cf3. In some examples, the cost function value (Cf) is equal to Cf1+Cf2+Cf3. In some examples, weights may be applied, such that Cf=w1*Cf1+w2*Cf2+w3*Cf3, 0<wi<1 and 0≤Cfi≤1. The weights (wi) may be pre-programmed. To complete the ER loop, processing circuitry 102 may determine which of the estimated shape model generates a Cf value that satisfies a threshold (e.g., less than threshold including minimized).

A first sub-cost function value (Cf1) is based on distances and orientation between the estimated shape models (e.g., generated based on different values for bi) and the first order shape generated by the ICP loop. For instance, for each of the estimated shape models, processing circuitry 102 may determine a Cf1 value. The equation for Cf1 may the same as the equation used for the ICP loop.

For example, Cf1=Σnorm(pₛ−pₜ)²+w*norm(nₛ−nₜ)²/N. However, in this case, pₛ and nₛ are for points and vectors of those points on each of the estimated shape models, and pₜ and nₜ are for points and vectors of those point on the first order shape. N refers to the number of points on the first order shape and on each of the estimated shape models.

Processing circuitry 102 may determine the value for Cf1 using the above example techniques described for the ICP loop. For example, for a first estimated shape model, processing circuitry 102 may determine a value of Cf1 (e.g., first Cf1), for a second estimated shape model, processing circuitry 102 may determine a value for Cf1 (e.g., second Cf1), and so forth. In this case, processing circuitry 102 may not perform any translation or rotation of any of the estimated shape models but utilize the calculation of Cf1 for each of the estimated shape models to select one of the estimated shape models.

The value of Cf1 is one of the sub-cost function values used to determine the cost function value of the ER loop. In some examples, it may be possible to determine the estimated shape model that minimizes the Cf1 value and end the ER loop. However, simply minimizing the difference (e.g., distance of points) between the output of the ICP loop and the estimated shapes generated from shape model 106 may not be sufficient to determine the pre-morbid characterization of the patient anatomy. For example, there may be logical constraints on the location of the shape generated by the ICP loop (e.g., the first order shape after the initial conclusion of the ICP loop) relative to the patient's body and minimizing the difference (e.g., distances) between the first order shape and the estimated shapes may possibly violate such logical constraints. For example, the sub-cost function value, Cf1, can be non-convex for some cases and therefore may lead to a local minimum that is incorrect. With additional sub-cost function values, processing circuitry 102 may correct for the non-convexity of the total cost function value, Cf.

For example, when predicting the pre-morbid characterization of the glenoid, the glenoid of the estimated shape models should not be more medialized than the current patient glenoid. In this disclosure, medialized or medial means towards the center of the patient's body. When the patient suffers injury or disease at the glenoid, the bone erosion may cause the pathological glenoid to be more medial (e.g., shift closer to the middle of the patient's body) than prior to the injury or aliment. Therefore, a glenoid on an instance of one of the estimated shape models that is more medial than the current glenoid is more than likely not a proper estimate of the pre-morbid patient anatomy. Again, the injury or disease may have caused the glenoid to become more medial, and therefore, if one of the estimated shape models includes a glenoid that is more medial than the current position of the glenoid, the instance of the estimated shape model may not have the pre-morbid characteristics of the patient anatomy.

That is, assume that a first estimated shape model, generated from shape model 106, minimized the value of Cf1 based on distances and orientation between the first estimated shape model and the first order shape generated by the ICP loop. In this example, if the glenoid of the first estimated shape model is more medial than the current position of the glenoid, the first estimated shape model may not be the correct or best estimate of the pre-morbid shape of the pathological anatomy.

To ensure that medialized instances of the estimated shape models are not used to determine the pre-morbid characterization, processing circuitry 102 may determine the value of Cf2. The value of Cf2 indicates whether the estimated shape model is more or less medial than the patient anatomy. That is, a second example sub-cost function value is Cf2. The value of Cf2 is a measure of constraints on medialization of anatomy.

To determine the value of Cf2, processing circuitry 102 may determine a threshold point (pₜₕ) laying on transverse axis 134 of the patient anatomy (e.g., pathological scapula). The point pₜₕ represents a threshold of the glenoid medialization that should be crossed by an instance of the intermediate shape model used to determine the pre-morbid characterizations. FIGS. 15A and 15B illustrate example ways in which to determine pₜₕ. For example, processing circuitry 102 may divide image data from scans 108 of the glenoid into four quarters of interest (e.g., superior, posterior, inferior, and anterior).

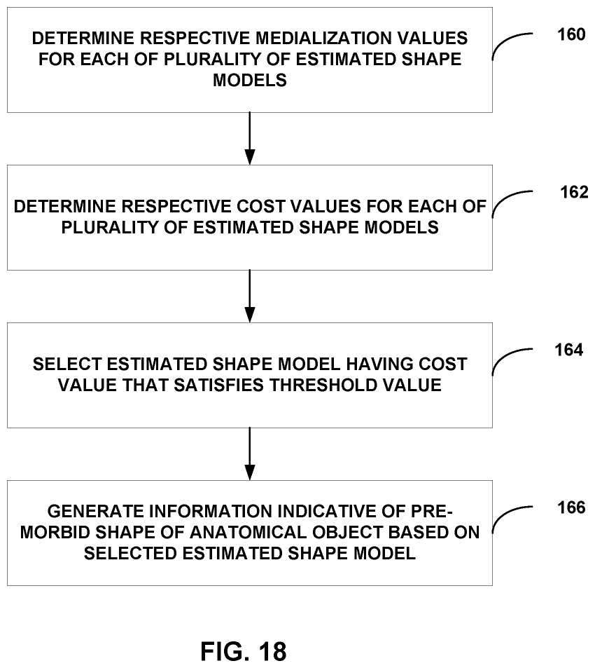

FIGS. 17A and 17B are conceptual diagrams illustrating portions of a glenoid for determining parameters of a cost function used to determine a pre-morbid shape of anatomy of a patient. As described in more detail, with the illustrated portions of the glenoid in FIGS. 17A and 17B, processing circuitry 102 may determine a value of pₜₕ. Processing circuitry 102 may also determine medialization values for each of the estimated shape models (e.g., generated from shape model 106 using values for bi and based on the eigenvalues and eigenvectors as described above). Based on the value of pₜₕ and the medialization values, processing circuity 102 may determine a sub-cost value for Cf2.

FIG. 17A illustrates transverse axis 134 (e.g., as described above) that divides a glenoid surface into anterior side 142 and posterior side 144. FIG. 17B illustrates transverse axis 134 that divides a glenoid surface into superior side 150 and inferior side 152. For instance, referring back to FIG. 8, processing circuitry 102 determined transverse axis 134. Then, processing circuitry 102 may determine anterior side 142, posterior side 144, superior side 150, and inferior side 152 based on axial or sagittal cuts through scapula 118. The result may be the example illustrated in FIGS. 17A and 17B.

Processing circuitry 102 may project points on the glenoid surface for each of the portions (e.g., anterior and posterior sides of FIG. 17A and superior and inferior sides of FIG. 17B) to transverse axis 134 and determine a center of mass of the projected points of each portion. Point projection onto a line is the closest point on that line. The line and the vector defined by the point and its projection are perpendicular. This can be calculated by choosing a random point on the line which forms a hypotenuse with the point to be projected. Using trigonometry, the projection is the hypotenuse length multiplied by the cosine of the angle defined by the hypotenuse and the line. The center of mass is the mean point of the projected points on that line and can also be considered as the barycenter.

For example, point 146 in FIG. 17A is an example of the center of mass of projected points for anterior side 142. Point 148 in FIG. 17A is an example of the center of mass of projected points for posterior side 144. Point 154 in FIG. 17B is an example of the center of mass of projected points for superior side 150. Point 156 in FIG. 17B is an example of the center of mass of projected points for inferior side 152.

In one or more examples, processing circuitry 102 may determine a most lateral barycenter point (e.g., the point that is furthest away from the center of the patient's body) from points 146, 148, 154, and 156. Processing circuitry 102 may set the most lateral barycenter point as the threshold point pm. Processing circuitry 102 may also determine the most lateral quarter as Qₜₕ. In some examples, the most lateral barycenter point need not necessarily be in the most lateral quarter. For example, assume that the point 156 is the most lateral barycenter point (e.g., threshold point pₜₕ). In this example, the most lateral quarter (Qₜₕ) may be inferior side 152 if the most lateral barycenter point were in the most lateral quarter. However, it is possible that the most lateral barycenter point is not in the most lateral quarter.

Processing circuitry 102 may determine a corresponding quarter in the instance of the estimated shape models with the Qth quarter (e.g., most lateral quarter). Processing circuitry 102 may project the points of the quarter of the instance of the estimated shape models to transverse axis 134. For example, for each of the estimated shape models, processing circuitry 102 may determine points like points 146, 148, 154, and 156 for each of the estimated shape models. However, because of the different medialization of the estimated shape models (e.g., due to the different values of bi used to generate the estimated shape models), the respective projected points may be at different locations on the transverse axis 134.

For example, processing circuitry 102 may determine an anchor point on transverse axis 134. This anchor point is in the same location on transverse axis 134 for each of the estimated shape models. Processing circuitry 102 may determine distances of the projected points to the anchor point. In some examples, pₜₕ may be the anchor point.

Processing circuitry 102 may determine the distance of the projected points to pₜₕ. Processing circuitry 102 may determine an average of the distances of the projected points as a value equal to d_{med}.

If the value of d_{med} is zero or positive, it means that the instance of the estimated shape model is more medial than the current patient anatomy (e.g., glenoid and scapula), and therefore not a good predictor for the pre-morbid characteristics of the patient anatomy. If the value of d_{med} is negative, it means that the instance of the estimated shape model is less medial than the current patient anatomy, and therefore may be a possible predictor for the pre-morbid characteristics of the patient anatomy.

In some examples, processing circuitry 102 may set Cf2 equal to a value based on d_{med} (e.g., Cf2 is a function of d_{med}). For example, the function used to calculate Cf2 based on d_{med} may be an increasing function for values of d_{med} greater than zero and a decreasing function for values of d_{med} less than zero. As one example, if d_{med} is greater than or equal to 0, processing circuitry 102 may set Cf2 equal to d_{med} and set Cf2 equal to 0 if d_{med} is less than zero. In this way, if the instance of the estimated shape models is more medial than the current patient anatomy, the value of Cf2 is a positive number, and the overall cost value of the cost function will increase. Again, the cost function (Cf) is equal to Cf1 plus Cf2 plus Cf3 (and possibly updated with weights w1, w2, and w3), and if Cf2 is a positive number, the value of Cf will increase as compared to if the value of Cf2 is zero. Because processing circuitry 102 is determining cost value that satisfies a threshold (e.g., minimizing the cost function), having a positive value of Cf2 causes processing circuitry 102 to not use instances of the estimated shape models that are more medial than the current patient anatomy for determining the pre-morbid characteristics.

In some examples, the cost function value (Cf) may be based only on Cf1 and Cf2. For instance, for the ER loop, processing circuitry 102 may determine which estimated shape model results in minimizing Cf. However, it may be possible that in such cases that the estimated shape model is one with high variation (e.g., has a lower probability of representing pre-morbid characteristics in the general population). Accordingly, in some examples, for the ER loop, processing circuitry 102 may determine a parameter weighting value (e.g., Cf3). The parameter weighting value weights more likely estimated shape models as having a higher probability of being a representation of the pre-morbid anatomy and weights less like estimated shape models as having a lower probability of being a representation of the pre-morbid anatomy.

For example, in some examples, processing circuitry 102 may also determine a sub-cost function value, Cf3, that is used to penalize against more complex solutions within an increasing variance in the data errors. For instance, processing circuitry 102 may determine a gradient smoothing term using eigenvalues to penalize the participating modes of variation according to the following equation.

$$Cf 3 = {\sum }_{l}^{N} \frac{{\sum }_{j}^{i} {λ}_{j}}{{\sum }_{j}^{N} λ j} \times {b}_{i}^{2}$$

In the above equation, N is the number of modes used to build an instance of the estimated shape model. Cf3 may not be necessary in all examples. For example, in the equation for Cf3, if an instance of the estimated shape model has many modes, then there will be a larger number of values to sum together, as compared to if the instance of the estimated shape model had fewer modes. Therefore, the result of the sum (e.g., value of Cf3) will be greater for those estimated shape models having larger modes than those estimated shape models having fewer modes. Accordingly, a larger value of Cf3 will cause the cost function value, Cf, to be bigger than a smaller value of Cf3. Because processing circuitry 102 may be minimizing the value of Cf, estimated shape models having a larger value of Cf3 are less likely to be determined as the pre-morbid shape of the patient anatomy as compared to estimated shape models having a smaller value of Cf3.

In general, Cf3 is composed of two terms: a coarse or hard term and a fine or smooth one. Eigenvalues are the diagonal positive values of the decomposition matrix. Their order reflects the occurrence of the corresponding eigenvectors into the database. Means that first eigenvalues represent the most “important” or frequent variation in the database, while the last eigenvalues represent the least frequent ones (usually representing noise). In the coarse term of Cf3, processing circuitry 102 may drop those eigenvectors that participate to the least important K % of the database variance (i.e. bi=0 if lambda(i)>q where vi for i<=q represents (100−K)% of the database variance). In the fine term of Cf3, processing circuitry 102 may gradually disadvantage higher eigenvectors. As a result the algorithm avoids complex or noisy solutions. In general, the terminology “smooth” is used to indicate addition of aa regularization term to an optimization method.

Processing circuitry 102 may determine the estimated shape model for which w1*Cf1+w2*Cf2+w3*Cf3 is less than a threshold (e.g., minimized). This estimated shape model is a first order shape model for the first iteration through the global loop.

For example, for the first iteration through the global loop, for the ER loop, assume processing circuitry 102 determines the following estimated shape models based on shape model 106 and different values of bi. For example, a first estimated shape model may be s11, where s11 is equal to s′+Σᵢ b11ᵢ\sqrt{λᵢ}×vᵢ, where s′ is shape model 106, λᵢ is the eigenvalues used to determine shape model 106, and vᵢ is the eigenvectors used to determine shape model 106. In this example, b11ᵢ is a first weighting parameter. Processing circuitry 102 may determine a second estimated shape model (e.g. s12), where s12 is equal to s′+Σᵢ b12ᵢ\sqrt{λᵢ}×vᵢ, where b12ᵢ is a second weighting parameter. In this manner, processing circuitry 102 may determine a plurality of estimated shape models (e.g., s11, s12, s13, and so forth).

For each of the estimated shape model, processing circuitry 102 may determine a value of Cf1, Cf2, and Cf3. Again, not all of Cf1, Cf2, and Cf3 are needed. For example, processing circuitry 102 may determine s11_Cf1 based on estimated shape model s11 and the first order shape generated by the ICP loop. Processing circuitry 102 may determine s12_Cf1 based on estimated shape model s12 and the first order shape generated by the ICP loop, and so forth. Processing circuitry 102 may determine s11_Cf2, s12_Cf3, and so forth as described above for the example techniques to determine Cf2. Similarly, processing circuitry 102 may determine s11_Cf3, s12_Cf3, and so forth as described above for the example techniques to determine Cf3.

Processing circuitry 102 may determine s11_Cf as: w1*s11_Cf1+w2*s11_Cf2+w3*s11_Cf3, determine s12_Cf as: w1*s12_Cf1+w2*s12_Cf2+w3*s13_Cf3, and so forth. The weights applied to the Cf1, Cf2, and Cf3 may be different for each of s11, s12, s13, and so forth, or the weights may be the same. Processing circuitry 102 may determine which one of s11_Cf, s12_Cf, s13_Cf, and so forth is the minimum (or possibly less than a threshold). Assume that s12_Cf is the minimum. In this example, processing circuitry 102 may determine estimated shape model s12 as the result of the ER loop, which for the first iteration of the global loop is the first order shape model.

This may conclude the first iteration of the global loop. For example, in the first iteration of the global loop, the ICP loop generated a first order shape and the ER loop generated a first order shape model. Then, for the second iteration of the global loop, the input to the ICP loop is the first order shape model generated from the previous ER loop and the first order shape generated from the previous ICP loop. In the ICP loop for the second iteration of the global loop, processing circuitry 102, based on the first order shape model and the first order shape, generates a second order shape. The second order shape is an input to the ER loop in the second iteration of the global loop. Also, in the ER loop, in the second iteration of the global loop, processing circuitry 102 may determine estimated shape models (e.g., s21, s22, s23, and so forth) based on shape model 106 and/or based on the first order shape model (e.g., s12 in this example). The output of the ER loop may be a second order shape model, and this may conclude the second iteration of the global loop.

This process repeats until processing circuitry 102 determines an instance of the estimated shape model that minimizes the Cf value. For instance, assume that after the first iteration of the global loop, processing circuitry 102 outputted estimated shape model s12 having cost value of s12_Cf. After the second iteration of the global loop, processing circuitry 102 outputted estimated shape model s23 having cost value of s23_Cf. After the third iteration of the global loop, processing circuitry 102 outputted estimated shape model s36 having cost value of s36_Cf. In this example, processing circuity 102 may determine which one of s12_Cf, s23_Cf, or s36_Cf is the minimum value, and determine the estimated shape associated with the minimum Cf value as the pre-morbid anatomy of the patient. For example, assume that s23_Cf was the minimum. In this example, processing circuity 102 may determine that estimated shape model s23 is represents the pre-morbid characteristics of the patient.

In the above examples, processing circuitry 102 may loop through the global loop until the value of Cf is minimized. However, in some examples, processing circuitry 102 may be configured to loop through the global loop for a set number of iterations. Processing circuitry 102 may then determine which one of the estimated shape models resulted in the minimum value of Cf. In some examples, processing circuitry 102 may loop through the global loop until the value of Cf is below a threshold.

As described above, processing circuitry 102 may determine a minimum for the cost function value, Cf. Minimizing the value of Cf or determining the minimum Cf value from a plurality of Cf values are two example ways in which to satisfy the cost function. There may be other ways in which to satisfy the cost function. As one example, satisfying the cost function may mean that the cost value of the cost function is less than a threshold. Also, it may be possible to reconfigure the cost function so that satisfying the cost function means maximizing the cost function. For instance, one of the factors in the cost function may be a distance between points of the estimated shape models and corresponding points in the output from the ICP loop. In some examples, processing circuitry 102 may minimize the distance between the estimated shape models and output from ICP loop and may generate a number that is inversely correlated to distance (e.g., the closer the distance, the larger the number processing circuitry 102 generates). In this example, to satisfy the cost function, processing circuitry 102 may maximize the cost value that that is inversely correlated to the distance. Therefore, although the examples are described with respect to minimizing as part of the global loop and the ICP loop, in some examples, to satisfy (e.g., optimize) the cost function, processing circuitry 102 may maximize the value of Cf. Such techniques are contemplated by this disclosure. For example, a cost value satisfying a threshold value means determining cost value is less than threshold value where cost value being less than threshold value is indicative of pre-morbid shape or greater than threshold value where cost value being greater than threshold value is indicative of pre-morbid shape.

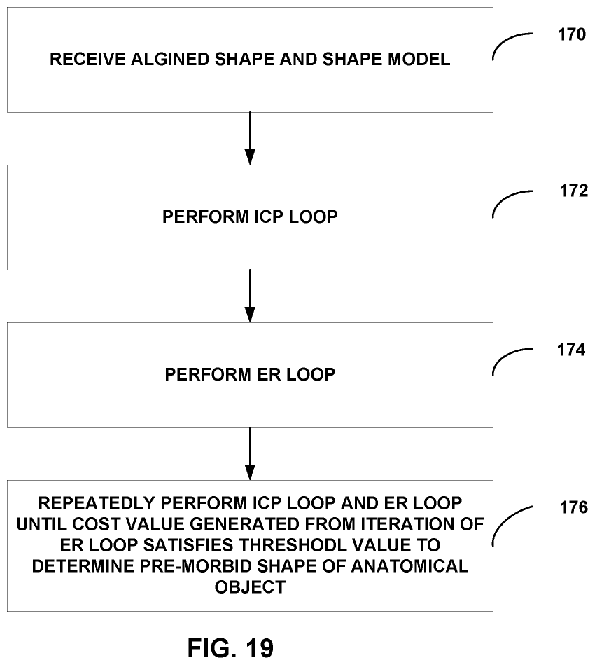

FIG. 18 is a flowchart illustrating an example method of operation in accordance with one or more example techniques described in this disclosure. For instance, FIG. 16 illustrates an example manner in which processing circuitry 102 may perform the ER loop. Memory 104 may store shape model 106, and information used to determine shape model 106 such as the eigenvalues and eigenvectors. In some examples, processing circuitry 102 may determine estimated shape models (e.g., by selecting different scaling factors (bᵢ) and store the estimated shape models in memory 104. In some examples, the estimated shape models may be pre-generated and stored in memory 104.

Processing circuitry 102 may determine respective medialization values (e.g., Cf2) for each of the plurality of estimated shape models (160). As described, the plurality of estimated shape models is generated from shape model 106. In some examples, the estimated shape models may be generated from the result of the ICP loop. Each of the medialization values is indicative of an amount by which each respective one of the estimated shape models is medialized relative to a current position of the anatomical object.

Processing circuitry 102 may determine respective cost values (Cf) for each of the plurality of estimated shape models based at least in part on the respective determined medialization values (Cf2) for each of the plurality of estimated shape models (162). In some examples, rather than just relying on Cf2, processing circuitry 102 may determine Cf1 and possibly Cf3. For example, processing circuitry 102 may determine a shape based on shape model 106 and an aligned shape generated from image data of the anatomical object. In this example, the shape determined based on shape model 106 and the aligned shape may be the result of the ICP loop. Processing circuitry 102 may determine respective distance and orientation difference values (Cf1) for each of the plurality of estimated shape models. Each of the respective distance and orientation difference values is indicative of a difference in distance and orientation between respective estimated shape models and the determined shape. Processing circuitry 102 may determine respective cost values for each of the plurality of estimated shape models based at least in part on the respective determined medialization values (Cf2) for each of the plurality of estimated shape models and the respective determined distance and orientation difference values (Cf1).

In some examples, processing circuitry 102 may determine respective weighting values (Cf3) based on a likelihood of each of the respective estimated shape models being representative of the pre-morbid shape of the anatomical object. In such examples, processing circuitry 102 may determine respective cost values (Cf) for each of the plurality of estimated shape models based at least in part on the respective determined medialization values (Cf2) for each of the plurality of estimated shape models and one or more of the respective determined distance and orientation difference values (Cf1) and the respective weighting values (Cf3).

To determining the respective weighting values (Cf3), processing circuitry 102 may be configured to perform

$${\sum }_{l}^{N} \frac{{\sum }_{j}^{i} {λ}_{j}}{{\sum }_{j}^{N} λ j} \times {b}_{i}^{2} .$$

N equals a number of modes used to build respective plurality of estimated shape models, λ equals eigenvalues of the shape model, and b equals a scaling factor used to determine respective plurality of estimated shape models.

To determine respective distance and orientation difference values (Cf1) for each of the plurality of estimated shape models, processing circuitry 102 may be configured to determine respective distances between corresponding points on respective estimated shape models and the determined shape, determine respective angular differences between normal vectors for the corresponding points on respective estimated shape models and the determined shape, and determine respective distance and orientation difference values based on the determine respective distances and the determined respective angular differences.

To determine respective medialization values for each of the plurality of estimated shape models, processing circuitry 102 may be configured to divide image data of the anatomical object into a plurality of portions, project points from each of the plurality of portions to a transverse axis, determine a threshold point from one of the projected points, determine a most lateral portion from the plurality of portion, determine a portion in each of the plurality of estimated shape models that corresponds to the determined most lateral position, project points from the determined portion in each of the plurality of estimated shape models to the transverse axis, determining distances from the projected points to the threshold point, and determine respective medialization values for each of the plurality of estimated shape models based on the determined distances.

Processing circuitry 102 may be configured to select an estimated shape model from the plurality of estimated shape models having (e.g., associated with) a cost value of the respective cost values that satisfies a function for the cost value (e.g., less than threshold value), including the example where the cost value is minimized (164). In this example, the estimated shape model having the cost value less than a threshold value is the result of the ER loop. Processing circuitry 102 may generate information indicative of the pre-morbid shape of the anatomical object based on the selected estimated shape model (166). For example, the output of the ER loop may be an input into the next operations of the ICP loop, and the ER loop and ICP loop may repeat until the cost value satisfies a threshold (e.g., less than threshold including minimized).

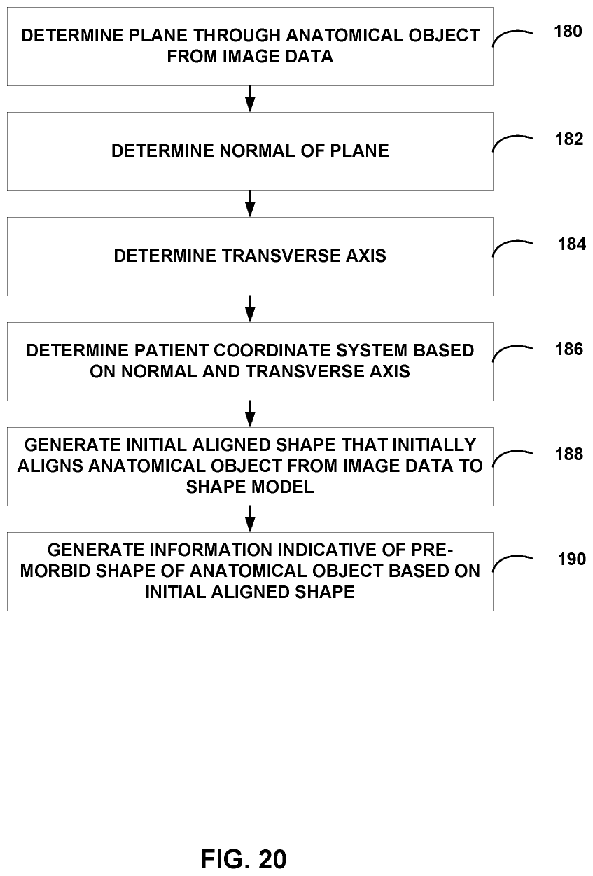

FIG. 19 is a flowchart illustrating an example method of operation in accordance with one or more example techniques described in this disclosure. For example, FIG. 19 illustrates an example of the global loop that includes the ICP loop and the ER loop. Processing circuitry 102 may receive an aligned shape and shape model 106 (170). Techniques to determine the aligned shape are described above and with FIG. 18.

Processing circuitry 102 perform the ICP loop (172). For example, to perform the ICP loop, processing circuitry 102 may be configured to modify the aligned shape, compare the modified aligned shaped to shape model 106, and determine a cost value based on the comparison. As also part of the ICP loop, processing circuitry 102 may repeat the modifying, comparing, and determining until the cost value satisfies a threshold value to generate a first order shape. The first order shape comprises the modified aligned shape associated with the cost value that satisfies the threshold value.

Processing circuitry 102 may perform the ER loop (174). To perform the ER loop, processing circuitry 102 may receive the first order shape and shape model 106. Processing circuitry 102 may determine respective cost values for each of a first set of plurality of estimated shape models based on the first order shape and respective plurality of estimated shape models. The plurality of estimated shape models may be generated based on the shape model 106. For example, processing circuitry 102 may determine value of Cf for each of the estimated shape models based on Cf1, Cf2, and/or Cf3 as described above. Processing circuitry 102 may determine a first estimated shape model of the plurality of estimated shape models associated with a cost value of the respective cost values that satisfies a first threshold value. The estimated shape model comprises a first order shape model.

The first order shape model from the ER loop may be fed back to the ICP loop and the ICP loop may utilize the first order shape model and the first order shape generated from the previous ICP loop to generate a second order shape. The second order shape is fed to the ER loop, and processing circuitry 102 utilizes the second order shape and the first order shape model and/or shape model 106 to generate a second order shape model that is fed back to the ICP loop, and this process repeats until the Cf value generated from the ER loop is below a threshold (e.g., minimized).

For example, processing circuitry 102 may repeatedly perform the ICP loop and the ER loop until a cost value generated from an iteration of the ER loop satisfies a second threshold value to determine the pre-morbid shape of the anatomical object (176). To repeatedly perform the ICP loop and the ER loop, processing circuitry 102 may be configured to receive, for the ICP loop, an order shape model generated by a previous ER loop and an order shape generated by a previous ICP loop, generate, by the ICP loop, a current order shape based on the order shape model and the order shape, receive, for the ER loop, the current order shape and at least one of shape model or the order shape model generated by the previous ER loop, generate a next set of plurality of estimated shape models based on at least one of the shape model or the order shape model generated by the previous ER loop, determine respective cost values for each of the second set of plurality of estimated shape models based on the order shape of the current order shape and respective second set of plurality of estimated shape models, and determine a current estimated shape model of the second set of plurality of estimated shape models associated with a cost value of the respective cost values that satisfies the first threshold value. The current estimated shape model is a current order shape model generated by the ER loop that is fed back to the ICP loop.

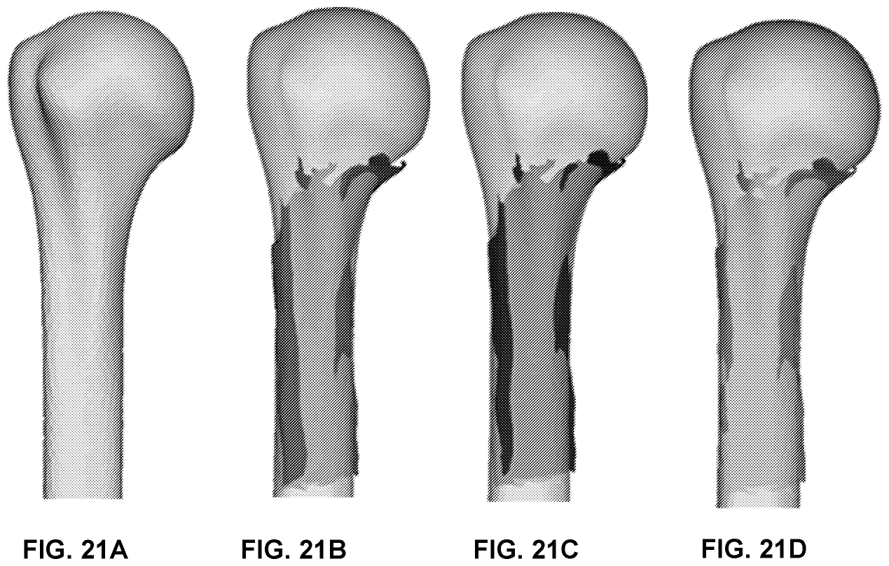

FIG. 20 is a flowchart illustrating an example method of operation in accordance with one or more example techniques described in this disclosure. For example, FIG. 20 illustrates an example manner in which to determine initial aligned shape 138 and aligned shape 140 based on a patient coordinate system.

Processing circuitry 102 may determine a plane 130 through the anatomical object from the image data. Plane 130 is a plane that is substantially through a middle of the anatomical object (180). Processing circuitry 102 may determine a normal (e.g., vector 132) of plane 130 (182). In addition, processing circuitry 102 may determine a transverse axis 134 through representations of sagittal cuts (e.g., as illustrated in FIGS. 9A, 9B, 10A, and 10B) through the anatomical object (184). Transverse axis 134 is orthogonal to the normal (e.g., vector 132).

Processing circuitry 102 may determine a patient coordinate system based on the normal (e.g., vector 132) and the transverse axis 134 (186). For example, processing circuitry 102 may determine the third axis, where vector 132 and transverse axis 134 form two of the three axes for defining 3D space, based on the vector 132 and transverse axis 134 as described above.

Processing circuitry 102 may generate an initial aligned shape that initially aligns the anatomical object from the image data to shape model 106 (188). For example, processing circuitry 102 may generate a transformation matrix and generate coordinates for anatomical objects that are in the coordinate system for shape model 106.

Processing circuitry 102 may generate information indicative of the pre-morbid shape of the anatomical object based on the initial aligned shape (190). For example, with initial aligned shape (e.g., initial aligned shape 138), processing circuitry 102 may iteratively adjust coordinates of the initial aligned shape to rotate the initial aligned shape until a distance between the initial aligned shape and the shape model satisfies a threshold value to generate an aligned shape 140. To generate information indicative of the pre-morbid shape of the anatomical object, processing circuitry 102 may generate information indicative of the pre-morbid shape based on the aligned shape 140. For example, aligned shape 140 may be an input into the global loop that includes the ICP loop and ER loop used to determine the pre-morbid shape.

FIGS. 21A-21H represent examples of initial alignment of a diaphysis with a statistical shape model (SSM) that includes a humerus and a scapula. The illustrated examples show the SSM being fit to the humerus. Using the process described about, in some examples, processing circuity 102 uses an intermediate model based on the SSM fit to the humerus to generate a first estimate of the shape of the scapula. That is, processing circuitry 102 may be configured to perform humerus SSM fitting such that fitting the humerus to the SSM also generates a first estimate of the scapula. FIGS. 21A-21D represent a first example of initial alignment of the diaphysis with humerus SSM, and FIGS. 21-21H represent a second example of initial alignment of the diaphysis with humerus SSM. FIGS. 21A-21D show a patient with a proximal diaphysis radius smaller than the diaphysis radius of the SSM. FIG. 21F demonstrates the output of the simplex optimization, where the SSM size has been decreased. Initial translation and orientation were computed very accurately. Hence, translation and rotation optimizations adjusted the initial position only by 2 mm and 3 degrees respectively.

FIGS. 21E-21H show a patient that has a similar diaphysis radius with the humerus SSM. The simplex optimization does not almost change the size of the humerus. Initial vertical alignment was accurately estimated, and the vertical adjustment procedure did not change the height of the broken diaphysis. However, FIG. 21G shows the wrong diaphysis orientation. The optimization process rotated the diaphysis by 68 degrees and on FIG. 21H shows a correctly oriented broken diaphysis. Processing circuitry 102 is configured to fit a humerus SSM onto the diaphysis. Processing circuitry 102 is configured to perform shape optimization. More specifically, processing circuitry 102 applies the simplex minimization algorithm that seeks to find a combination of principal modes of variation that minimizes the RMS distance between the diaphysis and SSM.

Humerus shape estimation may be used to align a shape model (e.g., shape model 202 of FIG. 2 above) to image data of a humerus. FIGS. 22A-22C show examples of humerus shape estimation based on diaphysis shape. FIGS. 22A-22C show an application of the previously described algorithm on three different patients. Visual observation of the obtained results demonstrates that the estimated model shapes are very close to the available diaphysis portions. The obtained results also show that the algorithm with the same accuracy can process diaphyses of different lengths. Examples of humerus shape estimation with a long portion of available diaphysis shown in FIGS. 22A and 22B. FIG. 22C shows an example of a predicted humerus shape based on a short diaphysis portion.

The humeral head possesses one anatomical and one geometric peculiarity that enables and/or necessitates the creation of a specific algorithm for its reduction. First, the humeral head is the only fragment that is not attached to any muscle. This anatomical peculiarity enables humerus head displacement in any directions and over long distances in case of proximal humerus fracture. Second, the humeral head surface can be very accurately approximated with a sphere shape. This geometric peculiarity makes it very easy to compute the humeral head center and register it to the humerus SSM head center.

Using the geometric properties of the spherical humeral head, processing circuitry 102 may implement the following algorithm to register the humeral head to the humerus SSM atlas.
Humeral head detection:
   Determine humeral head center C_{HH} as the voxel intersected by the biggest number of surface normals as described above;
   Determine the humeral head among the broken fragments Fj, j ∈ M, M
      number of detected broken fragments;
         For each fragment Fj fit a sphere in the least square sense and store the sphere center Cj;
         For each Cj compute the Euclidean distance Dj=dist(Cj, C_{HH});
   The minimal distance Dmin=minj∈M {Dj} corresponds to the humeral head F_{Head}←Fj, j is the index that corresponds to the distance Dmin. C_{HH} corresponds to the detected head center;
Humerus SSM head center detection:
   Manually selected humeral head vertex IDs on the mean shape enable their locations on the humerus SSM for any principal modes configuration to be known;
   Sphere fitting to these points permits us to compute the humerus SSM head center Chssm;
Humeral head centers alignment:
   Define a transformation T_{HH} as a translation vector Vₜᵣ=Chssm−C_{HH};
   Apply the transformation T_{HH} to the humeral head: F_{HeadAtHSSM}←Tr(F_{Head}, T_{HH});
Humeral head reduction:
   After the previous head centers alignment the orientation of the humeral head might be incorrect. To find the correct orientation, processing circuitry 102 applies the simplex optimization algorithm. By the mean of humeral head rotations around its rotational center, the optimization process minimizes the RMS distance between broken humeral head and the humerus SSM surface.

Examples of the humeral head reduction are shown in FIGS. 28A-28D and 29A-29D. The broken humeral head fragment fits perfectly into the predicted humerus shape. The obtained visual results demonstrate a correct behavior of the described broken head reduction algorithm.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuity,” as used herein may refer to any of the foregoing structures or any other structure suitable for implementation of the techniques described herein.

Various examples have been described. These and other examples are within the scope of the following claims.

1. A method for determining a pre-morbid shape of an anatomical object, the method comprising:
receiving first image data of a first anatomical structure and second image data of a second anatomical structure, the first and second anatomical structures being anatomically related;
determining a first estimated shape model based on the first image data and a joint statistical shape model (SSM), the first estimated shape model including a first estimated shape of the first anatomical structure and a first estimated shape for the second anatomical structure and the joint SSM is a shape model of combined first and second anatomical structures;
determining a second estimated shape model based on the first shape model, the first image data, and the second image data, the second shape model including a second estimated shape of the first anatomical structure and a second estimated shape for the second anatomical structure; and
generating anatomical information indicative of the pre-morbid shape of at least the second anatomical structure based on the second shape model.

2. The method of claim 1, further comprising:
determining a first aligned shape generated from the first image data and the second image data; and
determining respective distance and orientation difference values for each of a plurality of first shape models generated from the joint SSM, wherein each of the respective distance and orientation difference values is indicative of a difference in distance and orientation between respective first shape models and the first aligned shape,
wherein determining the first shape model of the first anatomical structure comprises selecting the first shape model from the plurality of first shape models having a cost value of the respective cost values that satisfies a function for the cost value, the cost value being based on the respective distance and orientation difference values.

3. The method of claim 2, wherein each of a plurality of first shape models includes a representation of the first anatomical structure and a representation of the second anatomical structure.

4. The method of claim 1, wherein generating the second estimated shape further comprises morphing the first estimated shape to the first image data and the second image data.

5. The method of claim 1, wherein the second image data includes a pathological portion and a non-pathological portion.

6. The method of claim 1, wherein the first image includes a greater percentage of data representative of the first anatomical object than the percentage of data in the second image representative of the second anatomical object.

7. The method of claim 6, wherein the second image data does not provide information to generate an aligned shape for the second anatomical structure without a second estimated shape selected based on the first shape model.

8. The method of claim 1, wherein the anatomical information includes a visual representation of at least one of an estimated pre-morbid shape of a humerus or an estimated pre-morbid shape of a scapula.

9. The method of claim 1, further comprising, based on the anatomical information, generating a surgical plan to correct a pathological state of at least one of a humerus or a scapula.

10. The method of claim 1, further comprising, based on the anatomical information, generating a representation of the first anatomical structure and the second anatomical structure to be displayed via a mixed-reality headset.

11. An apparatus comprising:
memory storing a plurality of first shape models; and
processing circuitry configured to:
receive first image data of a first anatomical structure and second image data of a second anatomical structure, the first and second anatomical structures being anatomically related;
determine a first shape model based on the first image data and a joint statistical shape model (SSM), the first shape model including a first estimated shape of the first anatomical structure and a first estimated shape for the second anatomical structure and the joint SSM is a shape model of combined first and second anatomical structures;
determine a second shape model based on the first shape model, the first image data, and the second image data, the second shape model including a second estimated shape of the first anatomical structure and a second estimated shape for the second anatomical structure; and
generate anatomical information indicative of the pre-morbid shape of at least the second anatomical structure based on the second shape model.

12. The apparatus of claim 11, wherein the processing circuitry is further configured to:
determine a first aligned shape generated from the first image data and the second image data; and
determine respective distance and orientation difference values for each of a plurality of first shape models generated from the joint SSM, wherein each of the respective distance and orientation difference values is indicative of a difference in distance and orientation between respective first shape models and the first aligned shape,
wherein to determine the first shape model of the first anatomical structure, processing circuity is to select the first shape model from the plurality of first shape models having a cost value of the respective cost values that satisfies a function for the cost value, the cost value being based on the respective distance and orientation difference values.

13. The apparatus of claim 12, wherein each of a plurality of first shape models includes a representation of the first anatomical structure and a representation of the second anatomical structure.

14. The apparatus of claim 11, wherein generating the second estimated shape further comprises morphing the first estimated shape to the first image data and the second image data.

15. The apparatus of claim 11, wherein the second image data includes a pathological portion and a non-pathological portion.

16. The apparatus of claim 11, wherein the first image includes a greater percentage of data representative of the first anatomical object than the percentage of data in the second image representative of the second anatomical object.

17. The apparatus of claim 16, wherein the second image data does not provide information to generate an aligned shape for the second anatomical structure without a second estimated shape selected based on the first shape model.

18. The apparatus of claim 11, wherein the anatomical information includes a visual representation of at least one of an estimated pre-morbid shape of a humerus or an estimated pre-morbid shape of a scapula.

19. The apparatus of claim 11, wherein the processing circuitry is further configured to, based on the anatomical information, generate a surgical plan to correct a pathological state of at least one of a humerus or a scapula.

20. The apparatus of claim 11, wherein the processing circuitry is further configured to, based on the anatomical information, generate a representation of the first anatomical structure and the second anatomical structure to be displayed via a mixed-reality headset.

21. A tangible computer readable medium comprising instructions that, when executed, cause processing circuitry to:
receive first image data of a first anatomical structure and second image data of a second anatomical structure, the first and second anatomical structures being anatomically related;
determine a first shape model based on the first image data and a joint statistical shape model (SSM), the first shape model including a first estimated shape of the first anatomical structure and a first estimated shape for the second anatomical structure and the joint SSM is a shape model of combined first and second anatomical structures;
determine a second shape model based on the first shape model, the first image data, and the second image data, the second shape model including a second estimated shape of the first anatomical structure and a second estimated shape for the second anatomical structure; and
generate anatomical information indicative of the pre-morbid shape of at least the second anatomical structure based on the second shape model.

22. The computer readable medium of claim 21, wherein the instructions, when executed, further cause the processing circuitry to:
determining a first aligned shape generated from the first image data and the second image data; and
determining respective distance and orientation difference values for each of a plurality of first shape models generated from the joint SSM, wherein each of the respective distance and orientation difference values is indicative of a difference in distance and orientation between respective first shape models and the first aligned shape,
wherein determining the first shape model of the first anatomical structure comprises selecting the first shape model from the plurality of first shape models having a cost value of the respective cost values that satisfies a function for the cost value, the cost value being based on the respective distance and orientation difference values.
